(19) United States
(12) Patent Application Publication
(10) Pub. No.: US 2024/0059645 A1
(43) Pub. Date: Feb. 22, 2024

(54) A CURING AGENT, A PROCESS FOR PREPARATION AND APPLICATION THEREOF

(71) Applicants: Jayram Mangesh NADKARNI, Mumbai (IN); Prasad Jayram NADKARNI, Mumbai (IN)

(72) Inventor: DATTATRAYA PANDURANG BARDE, MUMBAI (IN)

(21) Appl. No.: 18/258,821

(22) PCT Filed: Dec. 9, 2021
(86) PCT No.: PCT/IB2021/061498

§ 371 Date: Jun. 22, 2023

(30) Foreign Application Priority Data
Dec. 24, 2020 (IN) 202021056213

(51) Int. Cl. C07C 233/36; C09D 163/00

(52) U.S. Cl. CPC C07C 233/36; C09D 163/00

(57) ABSTRACT

The present invention is directed to phenalkamide curing agent of general formula (I) and a process for preparation thereof. The present invention further directed to an epoxy paint composition based on phenalkamide curing agent of formula (I).

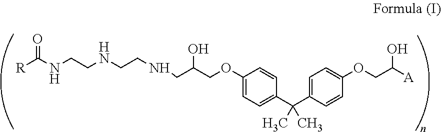

### BACKGROUND OF THE INVENTION

### Technical Field

The present invention relates to a curing agent of general formula (I). More particularly, the present invention relates to a phenalkamide curing agent of general formula (I) and a process for preparation thereof. The present invention further relates to a two component epoxy paint composition based on phenalkamide curing agent.

### Description of the Related Art

Epoxide compounds are known to be used with a curing agent and other additives to form a curable composition for various enduses. For example, in potting, infrastructure and hand lay-up composite applications, a curable composition for Such enduses generally requires a high reactivity (for example, a time to peak of less than (<) 150 minutes) and a low-exotherm (for example, a temperature of <125° C.) at the curing stage of a process for curing an epoxy resin composition to achieve effective production of a resulting thermoset product.

US20110020555A1 disclosed a two-component epoxy resin compositions are capable of faster cure at low ambient temperatures to quickly form non-sticky coatings and seals having a good appearance. The curing agent component is selected from phenalkamines. Preferably, the phenalkamines are prepared with cardanol, such as Cardolite™ 540, 541, and 541 LV phenalkamine hardeners, are preferred.

WO2006005723A1 discloses special amine compositions including (a) a polyetherdiamine, (b) a monoamine;and (c) a di- or tri-amine; and (d) an alkyphenol such as styrenated phenol. WO2006005723A1 also discloses that the amine compositions can be fonnulated with fast curing agents, such as Mannich bases.

Mostly epoxy coating composition is divided in two part one epoxy resin with more than one glycidyl group and other is curing agent an H donor with functional group such as amine, anhydride, carboxylic, sulphide etc. The category of epoxy curing agents contains broad range of chemicals and choice of curing agent varies depending on final performance of cured film required.

Phenalkamine are product from category mainly used in marine and industrial coating applications where high degree of performance like corrosion and chemical resistance is required. Conventional phenalkamine curing agents are produce by Mannich reaction of distilled Cashew nut shell liquid i. e. cardanol with polyamines and aldehyde. The phenalkamine curing agents based on renewable biomass has main advantage of low cost, fast cure at low temperature, good chemical & corrosion resistance, moisture tolerance, non-blushing properties, and good surface appearance. Also presence of long unsaturated side chain on cardanol gives low viscosity, good flexibility and surface tolerance. The presence of hydrophobicity due to side chain brings excellent water and sea water resistance. With all these advantages some of the main drawback of these curing agents are short pot life compared to polyamide, short overcoat window, dark Colour, and low Colour retention when expose to exterior atmosphere due to poor UV stability.

Polyamide series which are reaction product of higher polyamine and dimerized fatty acid to provide low to high viscosity products. Polyamide cured with epoxy resin at room temperature gives very good combination of hardness, flexibility with chemical and solvent resistance. Polyamide provides longer overcoat window, good pot life, low color and good UV stability, makes them suitable for top coat applications. However, main drawback of this product are high cost compare to phenalkamine, slow low temperature curing, poor acid, alkali, & chemical resistance.

So there is need to develop a new curing agent to overcome drawbacks of both Polyamide and Phenalkamine based curing agents with constructive performance from both categories.

### OBJECT OF THE INVENTION

Main objective of the present invention is to provide a phenalkamide curing agent of general formula (I).

Another objective of the present invention is to provide a process for the preparation of the phenalkamide curing agent of general formula (I).

Still another objective of the present invention to provide a paint composition based on phenalkamide curing agent.

### SUMMARY OF THE INVENTION

At least one specification heading is required. Please delete this heading section if it is not applicable to your application. For more information regarding the headings of the specification, please see MPEP 608.01(a).

In accordance with the above objectives, the present invention provides a phenalkamide curing agent of general formula (I) comprising:

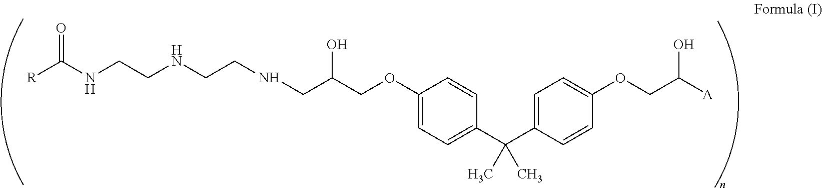

wherein, R is selected from hydrogen, or (un)substituted or substituted hydrogen, alkyl, alkenyl or alkynyl; or (un)substituted or substituted aryl, heteroalkyl, heteroaryl, arylalkyl, heteroarylalkyl; (un) substituted or substituted cycloalkyls, cycloalkenyl or cycloalkynyl; azo, amino, halo, nitro, cyano, hydroxyl, carbonyl, thiocarbonyl, carboxylic, alkoxy, carbamide, carbamate, hydrazine, sulfonyl, sulphide, thioether, sulphonamide, phosphates; A is selected from (un)substituted or substituted alkyl, alkenyl or alkynyl; or (un)substituted or substituted aryl, heteroalkyl, heteroaryl, arylalkyl, heteroarylalkyl; (un) substituted or substituted cycloalkyls, cycloalkenyl or cycloalkynyl; azo, amino, halo, nitro, cyano, hydroxyl, carbonyl, thiocarbonyl, carboxylic, alkoxy, carbamide, carbamate, hydrazine, sulfonyl, sulphide, thioether, sulphonamide, phosphates; n is selected from 1 to 4.

In another aspect, the present invention provides a process for preparation of the phenalkamide curing agent of general formula (I) comprising the steps of: charging a mixture of phenalkamine A or B and polyamide to obtain a reaction mixture; adding epoxy resin into the reaction mixture of step (a) over a period of 1 to 2 hours when temperature of the reaction mixture reached at a temperature 70° C. to 80° C. and followed by the stirring at the same temperature for 2 to 3 hours and cooling the reaction mixture of step (b) upto the 40° C. to 50° C. and adding the inert solvent to reduce the viscosity to afford the phenalkamide curing agent of general formula (I).

In still another aspect, the present invention provides a coating composition comprising
a) the phenalkamide curing agent of general formula (I); b) an epoxy resin having on average more than one glycidyl group per molecule and c) pigments.

### BRIEF DESCRIPTION OF DRAWINGS

FIG. 1: IR of PA1;

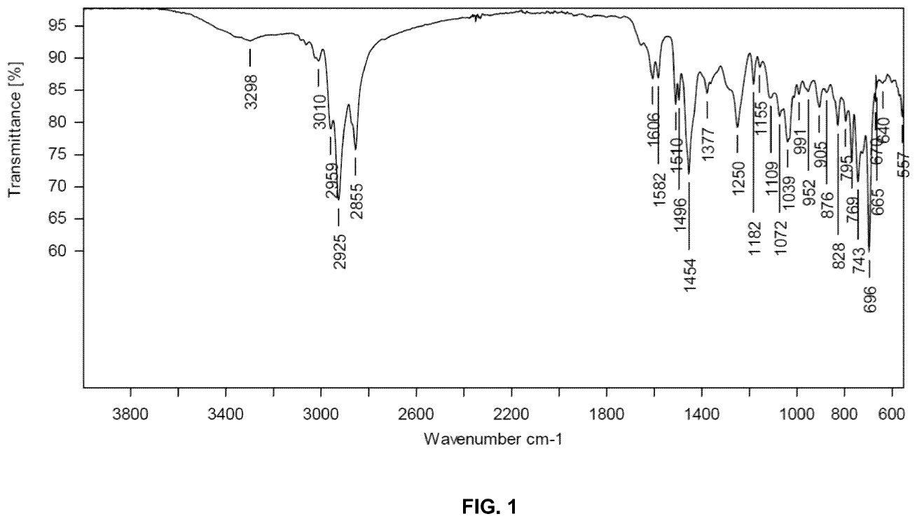

FIG. 2: 1H NMR of PA1;

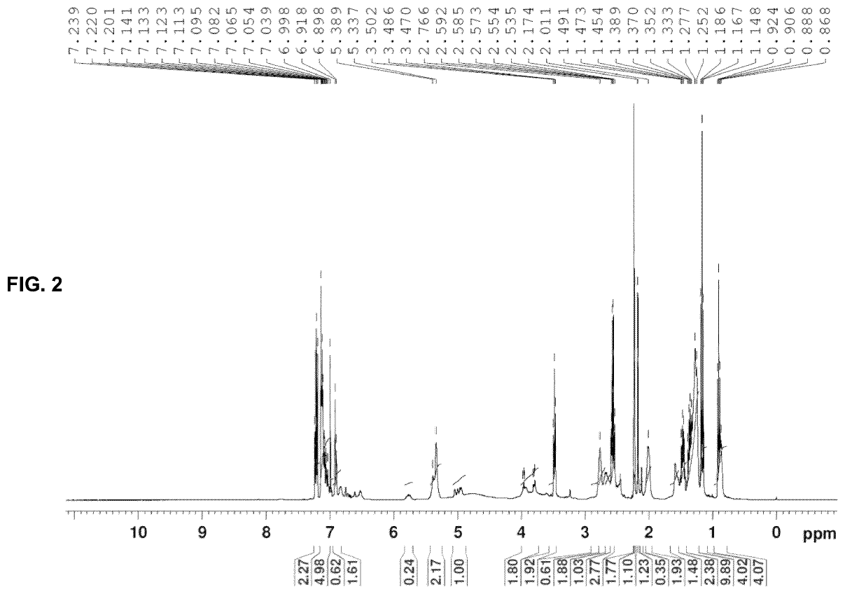

FIG. 3: 13C NMR of PA1;

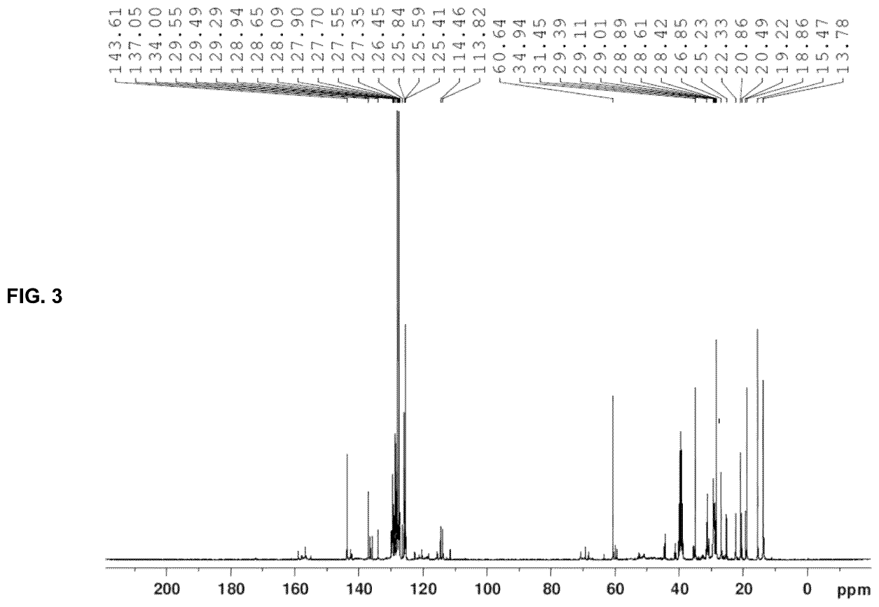

FIG. 4: IR of PA2;

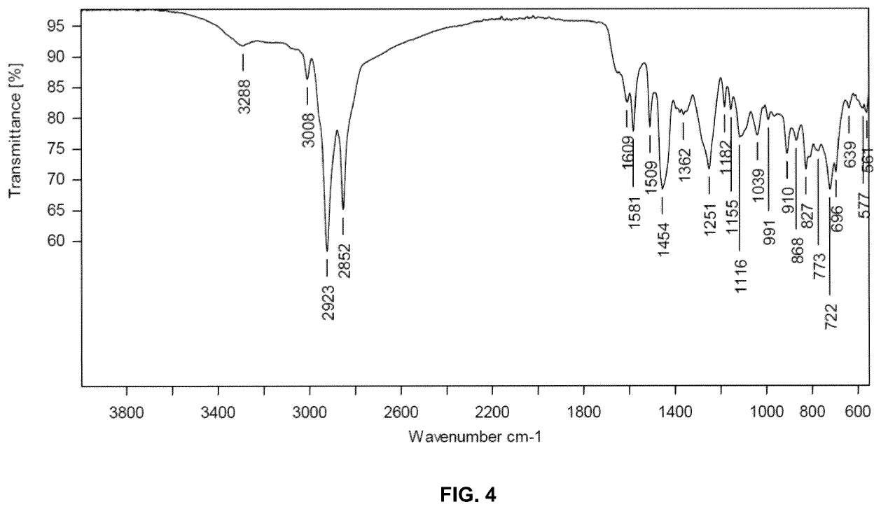

FIG. 5: 1H NMR of PA2; and

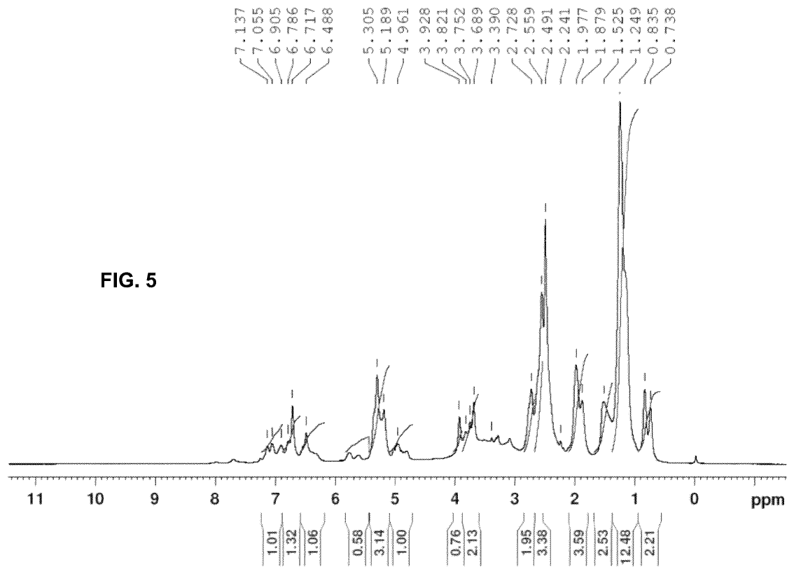

FIG. 6: 13C NMR of PA2.

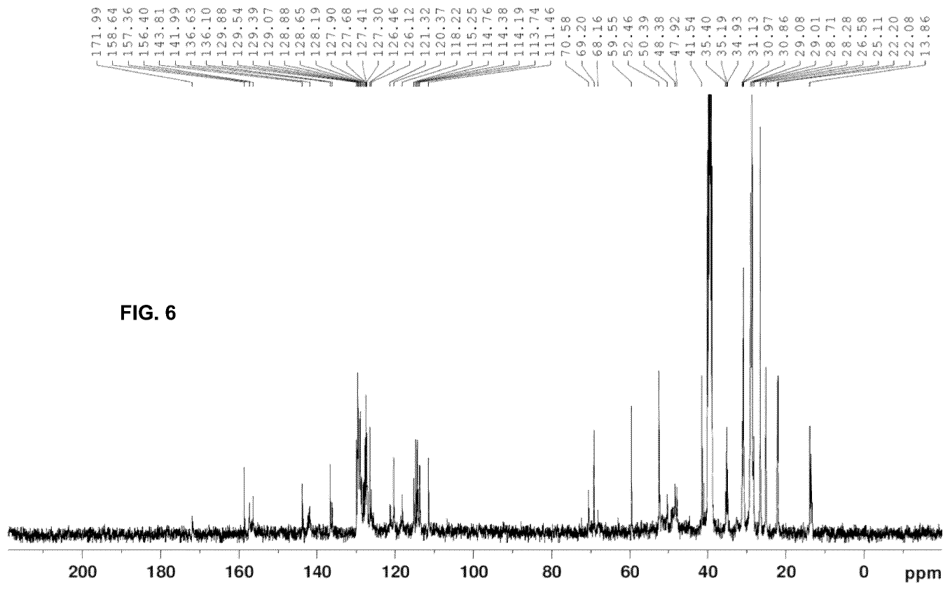

### DETAILED DESCRIPTION

The invention will now be described in detail in connection with certain preferred and optional embodiments, so that various aspects thereof may be more fully understood and appreciated.

Glossary:

With reference to the invention, the terms have the meaning as set forth below:

The term “alkyl” means a saturated straight or branched C₁-C₂₀hydrocarbon group which include substituted and unsubstituted alkyl groups methyl, ethyl, propyl, isopropyl, butyl, isobutyl, t-butyl, pentyl, isopentyl, neopentyl, hexyl, heptyl, octyl, 2-methyl-1-propyl, 2-chloro-2-propyl, 2-bromo-1-butyl, 2-methyl-3-butyl, 2,2-dimethyl-1-propyl, 2-methyl-1-pentyl, 3 -methyl-1-pentyl, 4-methyl-1-pentyl, 2-methyl-2-pentyl, 3-methyl-2-pentyl.

The term “alkenyl” means an unsaturated C₁-C₂₀alkyl group having at least one double bond which includes substituted and unsubstituted alkenyl groups vinyl, allyl, 1-propenyl, isopropenyl, 2-butenyl, 2-pentenyl, 2-hexenyl, and substituted C2-4 alkenyls.

The term “alkynyl” means an unsaturated C₁-C₂₀alkyl group having at least one triple bond which includes substituted and unsubstituted alkynyl groups such as ethynyl, propynyl, butynyl, pentynyl, hexynyl, methylpropynyl.

The term “aryl” means a carbocyclic single or multiple aromatic ring system which include phenyl, tolyl, anthracenyl, fluorenyl, indenyl, and naphthyl. The aromatic ring can be substituted at atleast one ring position with substituents that include, e.g., alkanoyl, alkoxy, alkyl, alkenyl, alkynyl, amido, amidino, amino, aryl, arylalkyl, azido, azo, carbamate, carbamide, carbonate, carbonyl, carboxy, cyano, cycloalkyl, ester, ether, formyl, halogen, haloalkyl, heteroaryl, heterocyclyl, hydrazine, hydroxyl, imino, ketone, nitro, sulfate, sulfide, sulfonamido, sulfonyl and thiocarbonyl. The term “aryl” also includes polycyclic ring systems having at least two cyclic rings in which at least two carbons are common to two adjoining rings (the rings are “fused rings”) wherein at least one of the rings is aromatic, e.g., the other cyclic rings can be cycloalkyls, cycloalkenyls, cycloalkynyls, aryls, and combinations thereof. The 2-chloro-1-methylbenzene, 1-chloro-4-methoxybenzene, 4-butoxyphenyl, 4-pentylphenyl, 4-isopropylphenyl, phenyl, 3-chloro-4-methylphenyl, 2-chloro-5-methoxyphenyl, phenyl, 3-chlorophenyl, 3-methoxyphenyl, 2-methoxyphenyl, Ph-COC₄H₉, N,N-dimethyl aniline, 4-chlorophenyl, 4-fluorophenyl, 4-butoxyphenyl, 4-methoxyphenyl, 4-pentylphenyl, 4-isopropylphenyl, p-tolyl, cyclohex-1-en-1-yl, 2-(hept-1-yn-1-yl), phenanthren-9-yl, 2-(naphthalen-2-yl) or 2-(naphthalen-1-yl).

The term “arylalkyl” means an aryl group having at least one alkyl substituent. Arylalkyl groups include substituted and unsubstituted arylalkyl groups including, e.g., arylalkyls having a monocyclic aromatic ring system in which the ring includes 6 carbon atoms.

The term “carbonyl” means the group —C(O)—R₉, where R₉ is selected from alkyl, alkenyl, alkynyl, alkoxy,amide, amidino, aryl, arylalkyl, carbamate, carbonyl, carboxy, cyano, cycloalkoxy, cycloalkyl, ether, halo, haloalkyl, heteroaryl, heterocyclyl, heterocycloalkyl, heterocyclylalkoxy, heterocyclyloxyalkyl, hydrogen, hydroxyl, hydroxyalkyl, hydrazine, azo, carbamide, imino, sulfide, and thiocarboxy.

The term “carboxy” means the group —COOH and its corresponding salts, e.g. —COONa.

The term “cyano” means the group —CN.

The terms “halo” and “halogen” mean fluorine, chlorine, bromine or iodine.

The term ‘nitro’ means the group —NO₂

The term “haloalkyl” means an alkyl group substituted with at least one halogen atom.

The term “heteroaryl” means a mono- or multi-cyclic aromatic ring system containing at least one heteroatom. Heteroaryls can also be fused to non-aromatic rings imidazolyl, indazolyl, indolizinyl, indolyl, isoindolyl, isoquinolinyl, isothiazolyl, isoxazolyl, naphthyridinyl, oxadiazolyl, oxazolyl, phenanthrenyl, phenanthridinyl, phenanthrolinyl, phenarsazinyl, phenazinyl, phenothiazinyl, phenoxazinyl, phthalazinyl, pteridinyl, purinyl, pyrazinyl, pyrazolyl, pyrazyl, pyridazinyl, pyridinyl, pyrimidilyl, pyrimidyl, pyrrolyl, quinolinyl, quinolizinyl, quinoxalinyl, quinoxaloyl, quinazolinyl, tetrazolyl, thiadiazolyl, thianthrenyl, thiazolyl, thienyl, thiophenyl, triazinyl, (1,2,3)- and (1,2,4)-triazolyl. The heteroaryl ring can be substituted at atleast one position with such substituents as described above including, e.g., alkanoyl, alkoxy, alkyl, alkenyl, alkynyl, amido, amidino, amino, aryl, arylalkyl, azido, azo, carbamate, carbamide, carbonate, carbonyl, carboxy, cyano, cycloalkyl, ester, ether, formyl, halogen, haloalkyl, heteroaryl, heterocyclyl, hydrazine, hydroxyl, imino, ketone, nitro, phosphate, phosphonato, phosphinato, sulfate, sulfide, sulfonamido, sulfonyl and thiocarbonyl.

The term ‘heteroarylalkyl’ means heteroaryl group having at least one alkyl substituent which may be substituted or unsubstituted.

The term ‘cycloalkyl’ means C₁ to C₈ saturated cyclic rings which may be substituted or unsubstituted.

The term cycloalkenyl' means C₁ to C₈ saturated cyclic rings having at least one double which may be substituted or unsubstituted.

The term cycloalkynyl' means C₁ to C₈ saturated cyclic rings having at least one triple bond which may be substituted or unsubstituted.

The term ‘amino’ means a group —NR₁₀R₁₁ wherein R₁₀ and R₁₁ independently represent hydrogen, alkyl, alkenyl, alkynyl, aryl, heteroaryl, cycloalkyl, halo, hydroxyl, carboxy or carbonyl.

The term ‘carbamate’ means the group —R₁₂OC(O)N(R₁₃)n wherein each R₁₃ represent independently alkyl, alkenyl, alkynyl, aryl, heteroaryl, cycloalkyl, halo, hydroxyl, cycloalkyl, heterocycycyl and ‘n’ is 1 or 2.

The term ‘carbamide’ means the group —N(R₁₄)—(CO)N(R₁₄)m where each R14 represent alkyl, alkenyl, alkynyl, aryl, heteroaryl, cycloalkyl, halo, hydroxyl, cycloalkyl or heterocycycyl and ‘m’ is 1 or 2.

The term ‘azido’ means the group —N₃.

The term “hydrazine” means the group —N(R₁₅)N(R₁₅)₂ where each R₁₅ independently represent alkyl, alkenyl, alkynyl, aryl, heteroaryl, cycloalkyl, halo, hydroxyl, cycloalkyl or heterocycle.

The term “phosphate” means the group —OP(O)(OR₁₆)₂ or its anions where each R₁₆ independently represent alkyl, alkenyl, alkynyl, aryl, heteroaryl, cycloalkyl, halo, hydroxyl, cycloalkyl or heterocyclyl.

The term “sulfate” means the group —OS(O)(OR₁₇)₂ or its anions where each R₁₇ independently represent alkyl, alkenyl, alkynyl, aryl, heteroaryl, cycloalkyl, halo, hydroxyl, cycloalkyl or heterocyclyl.

The terms “sulfonamide” mean a group having the structure —N(R₁₈)—S(O)₂—R₁₈— or —S(O)₂—N(R₁₈)x where each R₁₈ independently represent alkyl, alkenyl, alkynyl, aryl, heteroaryl, cycloalkyl, halo, hydroxyl, cycloalkyl or heterocyclyl; and ‘x’ is 1 or 2.

The term “sulfonyl” means a group having the structure R₁₉SO₂— where R₁₉ represent alkyl, alkenyl, alkynyl, aryl, heteroaryl, cycloalkyl, halo, hydroxyl, cycloalkyl or heterocyclyl.

The term “sulfide” and “thioether” means a group having the structure R₂₀S— where R₂₀ represent alkyl, alkenyl, alkynyl, aryl, heteroaryl, cycloalkyl, halo, hydroxyl, cycloalkyl or heterocyclyl.

The term “ether” means a group having the structure —R₂₁—O—R₂₁—, where each R₂₁ represent independently alkyl, aryl, cycloalkyl or heterocyclyl which may be optionally substituted.

The term amide means as an organic amide or a carboxamide, is a compound with the general formula R₂₂CNR₂₃R₂₄, where R₂₂, R₂₃, and R₂₄ represent organic groups or hydrogen atoms.

The chemical groups described herein can be substituted where valency of the atom permits substitution unless otherwise specified.

In an embodiment, the present invention provides a phenalkamide curing agent of general formula (I) represented as:

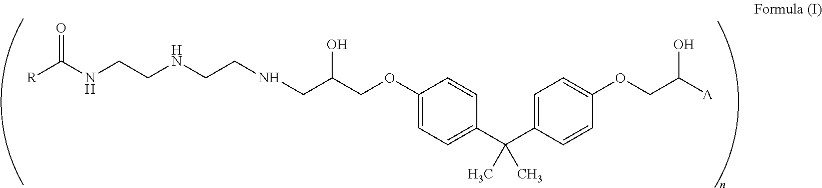

wherein,
R is selected from hydrogen, or (un)substituted or substituted hydrogen, alkyl, alkenyl or alkynyl; or (un)substituted or substituted aryl, heteroalkyl, heteroaryl, arylalkyl, heteroarylalkyl; (un) substituted or substituted cycloalkyls, cycloalkenyl or cycloalkynyl; azo, amino, halo, nitro, cyano, hydroxyl, carbonyl, thiocarbonyl, carboxylic, alkoxy, carbamide, carbamate, hydrazine, sulfonyl, sulphide, thioether, sulphonamide, phosphates;
A is selected from (un)substituted or substituted alkyl, alkenyl or alkynyl; or (un)substituted or substituted aryl, heteroalkyl, heteroaryl, arylalkyl, heteroarylalkyl; (un) substituted or substituted cycloalkyls, cycloalkenyl or cycloalkynyl; azo, amino, halo, nitro, cyano, hydroxyl, carbonyl, thiocarbonyl, carboxylic, alkoxy, carbamide, carbamate, hydrazine, sulfonyl, sulphide, thioether, sulphonamide or phosphates;
n is selected from 1 to 4.

In a preferred embodiment, the present invention provides a phenalkamide curing agent of general formula (I) comprising:

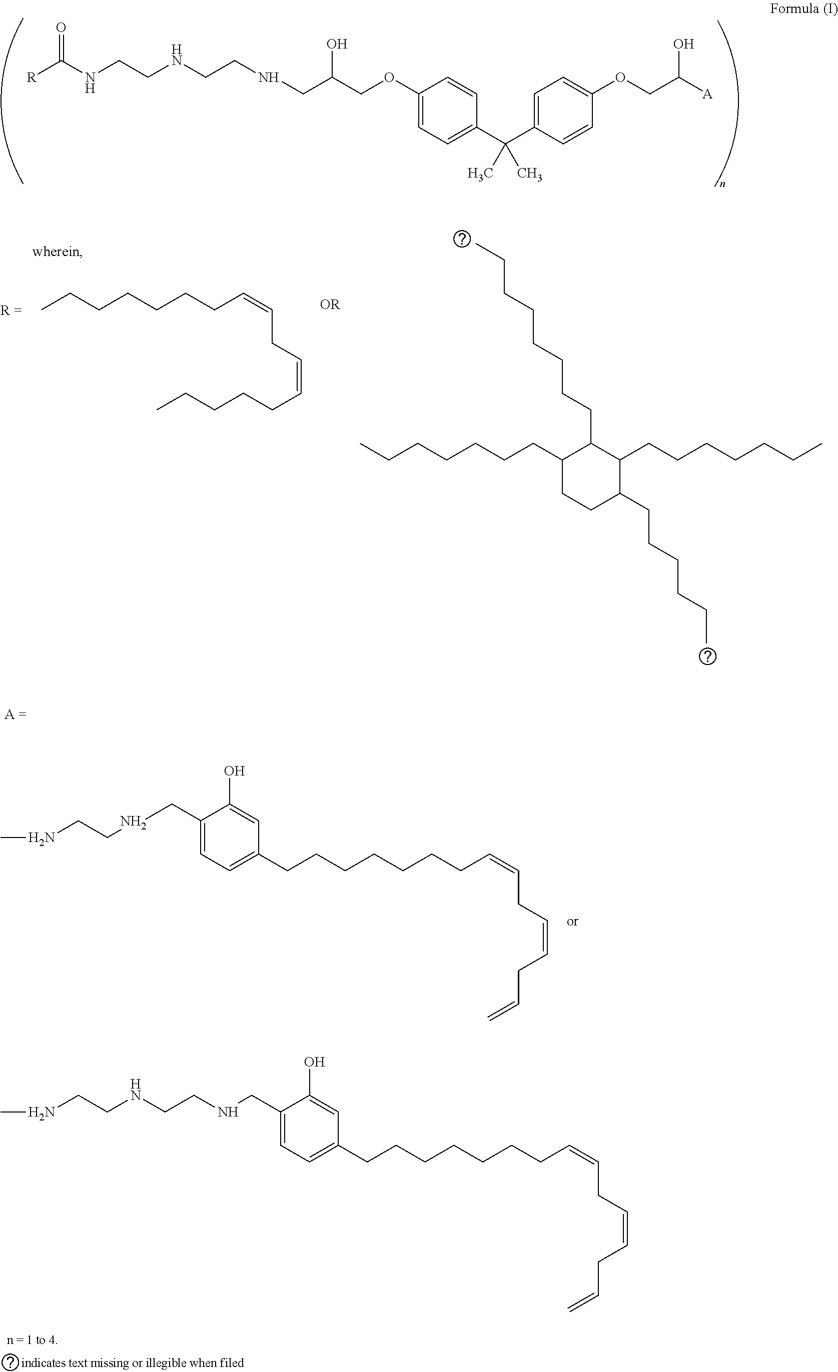

In another preferred embodiment, the phenalkamide curing agent of general formula (I) is selected from Phenalkamide PA-1 and Phenalkamide PA-2.

The Phenalkamide PA-1 is selected from

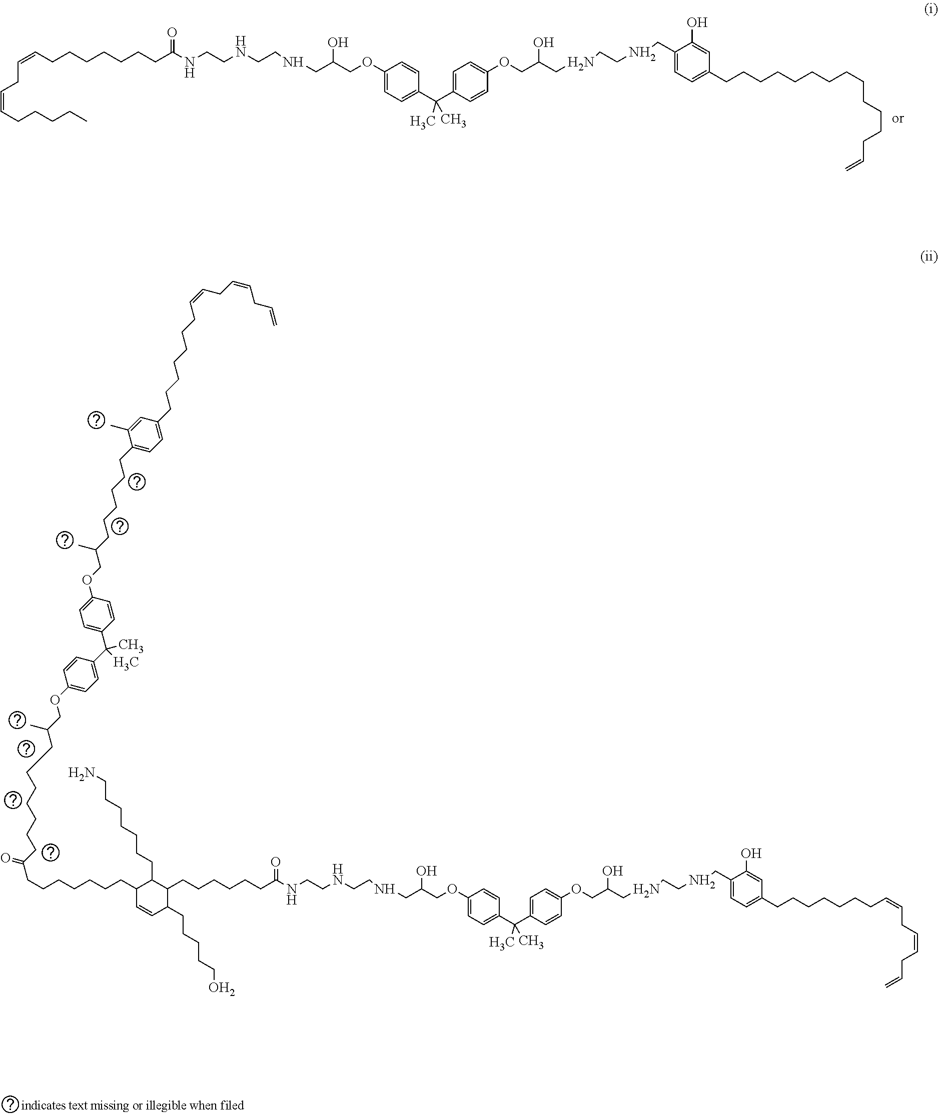 [SMILES: C=CCCCCCCCCCCCCCc1ccc(CNCCNCC(O)COc2ccc(C(C)(C)c3ccc(OCC(O)CNCCNCCNC(=O)CCCCCCC/C=C\C/C=C\CCCCC)cc3)cc2)c(O)c1 ; C=CC/C=C\C/C=C\CCCCCCCc1ccc(CCCCCCC(C)COc2ccc(C(C)(C)c3ccc(OCC(C)CCCCCCCCC(=O)CCCCCCCC4C=CC(CCCCCO)C(CCCCCCC(=O)NCCNCCNCC(O)COc5ccc(C(C)(C)c6ccc(OCC(O)CNCCNCc7ccc(CCCCCCC/C=C\C/C=C\CC=C)cc7O)cc6)cc5)C4CCCCCCCN)cc3)cc2)c(C)c1]

The Phenalkamide PA-2 is selected from

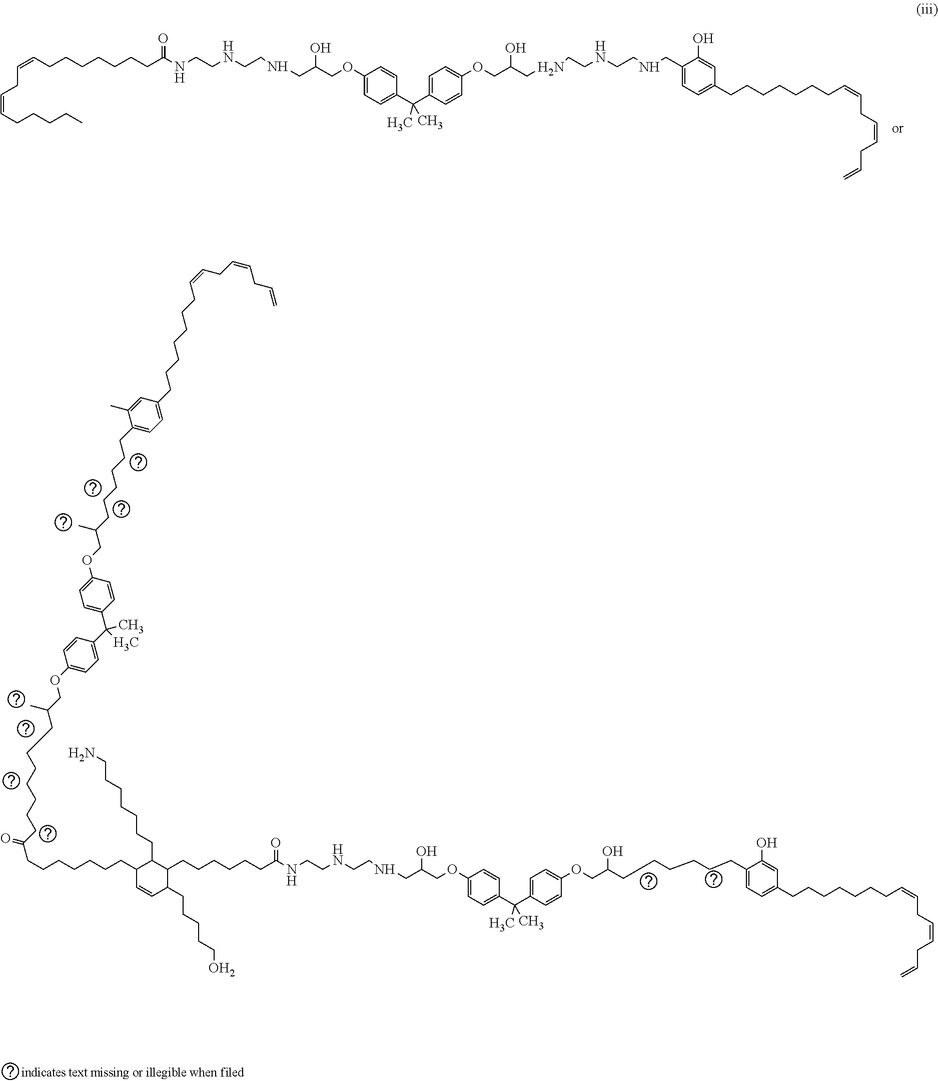 [SMILES: C=CC/C=C\C/C=C\CCCCCCCc1ccc(CNCCNCCNCC(O)COc2ccc(C(C)(C)c3ccc(OCC(O)CNCCNCCNC(=O)CCCCCCC/C=C\C/C=C\CCCCC)cc3)cc2)c(O)c1 ; C=CC/C=C\C/C=C\CCCCCCCc1ccc(CCCCCCC(C)COc2ccc(C(C)(C)c3ccc(OCC(C)CCCCCCCCC(=O)CCCCCCCC4C=CC(CCCCCO)C(CCCCCCC(=O)NCCNCCNCC(O)COc5ccc(C(C)(C)c6ccc(OCC(O)CCCCCCCc7ccc(CCCCCCC/C=C\C/C=C\CC=C)cc7O)cc6)cc5)C4CCCCCCCN)cc3)cc2)c(C)c1]

In another embodiment, the present invention provides a process for preparation of the phenalkamide curing agent of general formula (I) comprising the steps of:
charging a mixture of phenalkamine A or B and polyamide to obtain a reaction mixture;
adding epoxy resin into the reaction mixture of step (a) over a period of 1 to 2 hours when temperature of the reaction mixture reached at a temperature 70° C. to 80° C. and followed by the stirring at the same temperature for 2 to 3 hours and
cooling the reaction mixture of step (b) upto the 40° C. to 50° C. and adding the inert solvent to reduce the viscosity to afford the phenalkamide curing agent of general formula (I).

The above process is as shown in following scheme 1 and 2:

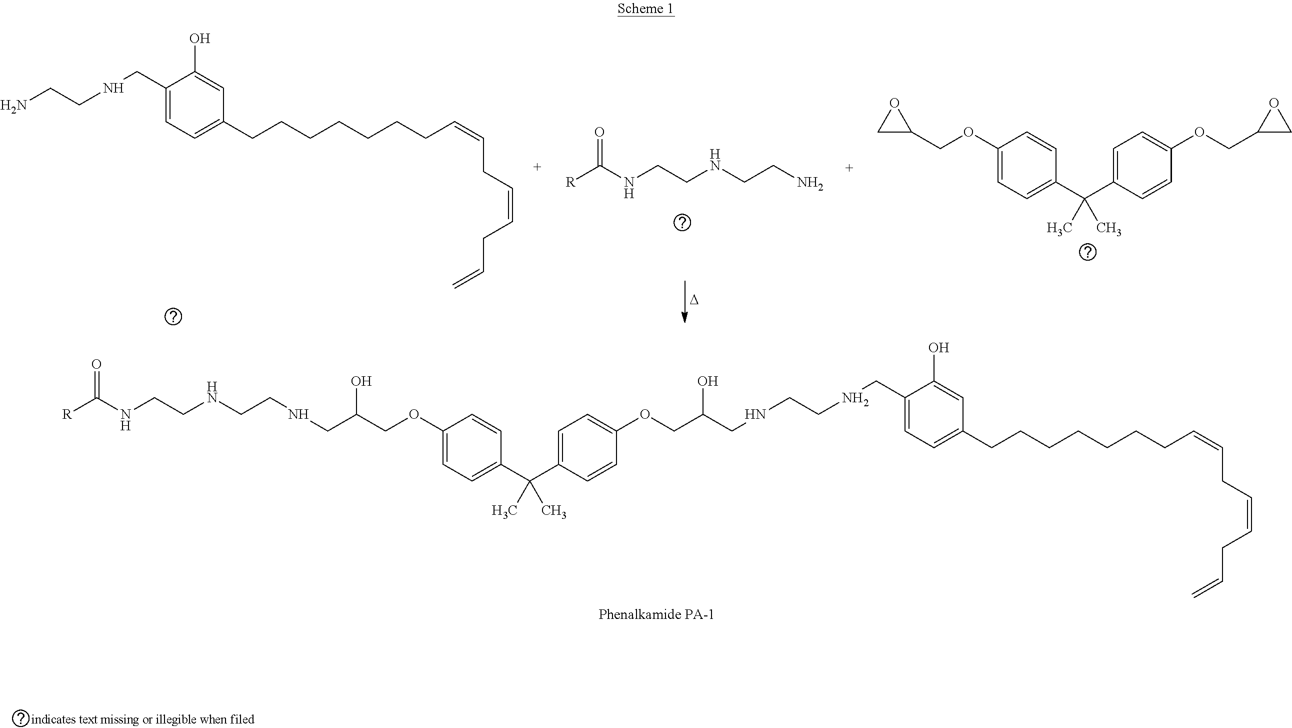

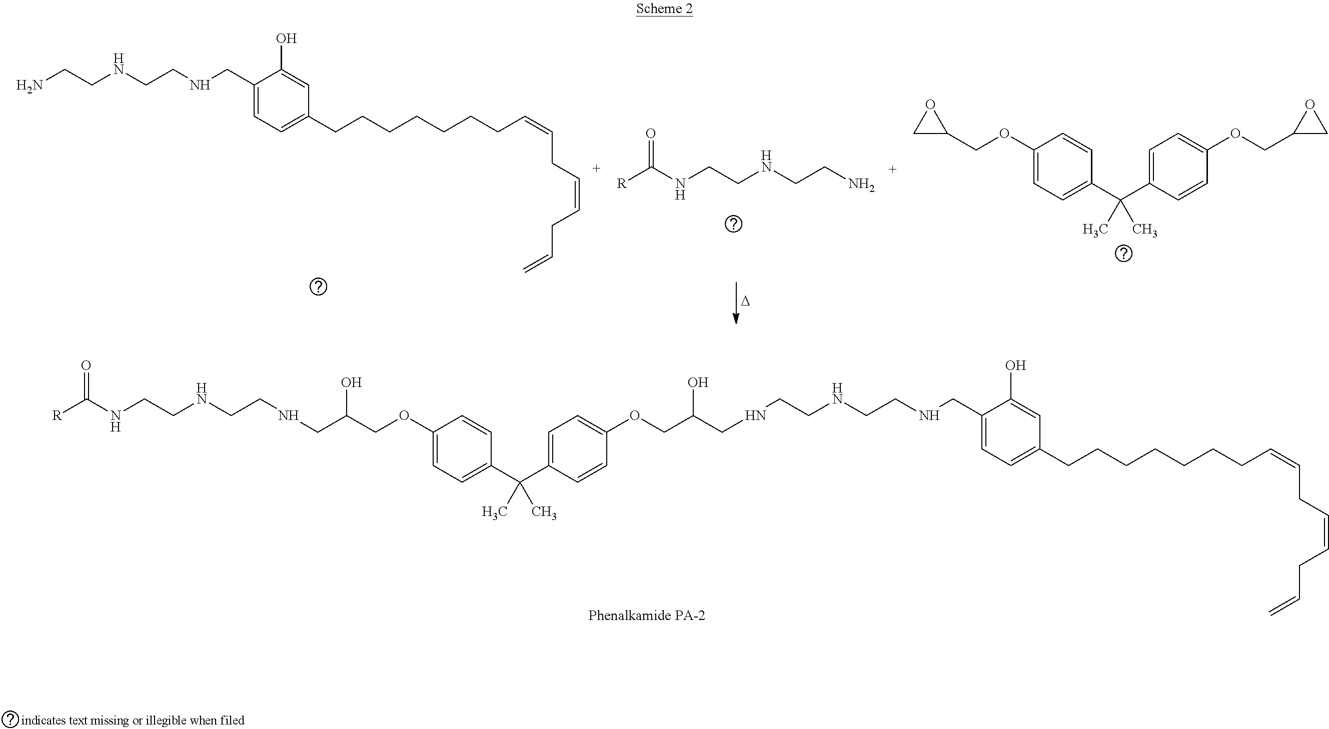

wherein,
R is selected from hydrogen, or (un)substituted or substituted hydrogen, alkyl, alkenyl or alkynyl; or (un)substituted or substituted aryl, heteroalkyl, heteroaryl, arylalkyl, heteroarylalkyl; (un) substituted or substituted cycloalkyls, cycloalkenyl or cycloalkynyl; azo, amino, halo, nitro, cyano, hydroxyl, carbonyl, thiocarbonyl, carboxylic, alkoxy, carbamide, carbamate, hydrazine, sulfonyl, sulphide, thioether, sulphonamide, phosphates;
preferably, R is selected from

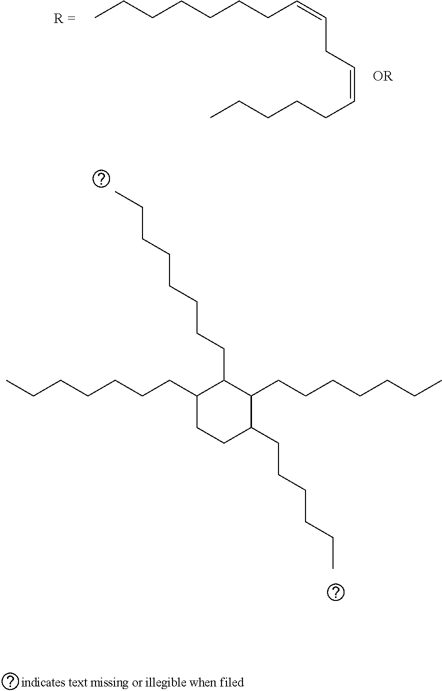

In a preferred embodiment, the phenalkamine A and phenalkamine B of step (a) are represented as below:

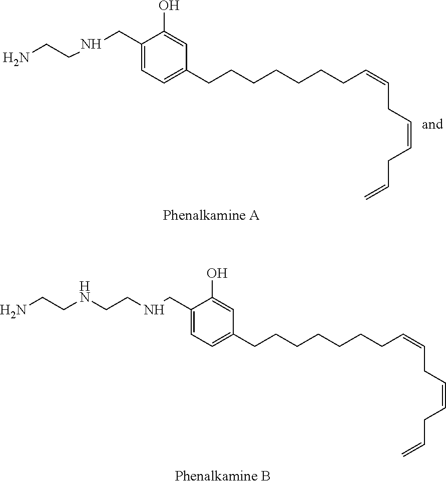 [SMILES: C=CC/C=C\C/C=C\CCCCCCCc1ccc(CNCCN)c(O)c1 ; C=CC/C=C\C/C=C\CCCCCCCc1ccc(CNCCNCCN)c(O)c1]

In another preferred embodiment, the polyamide of step (a) is represented as below:

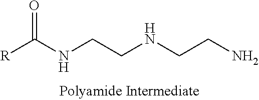

wherein R is selected from
R is selected from hydrogen, or (un)substituted or substituted hydrogen, alkyl, alkenyl or alkynyl; or (un)substituted or substituted aryl, heteroalkyl, heteroaryl, arylalkyl, heteroarylalkyl; (un) substituted or substituted cycloalkyls, cycloalkenyl or cycloalkynyl; azo, amino, halo, nitro, cyano, hydroxyl, carbonyl, thiocarbonyl, carboxylic, alkoxy, carbamide, carbamate, hydrazine, sulfonyl, sulphide, thioether, sulphonamide, phosphates;
preferably, R is selected from

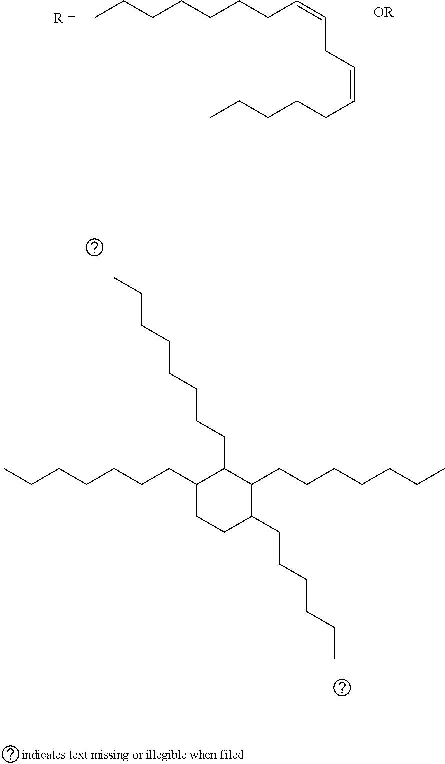

The epoxy resin of step (b) contain atleast two glycidyl ether groups. Resin backbone may be saturated or unsaturated, aliphatic, cycloaliphatic, aromatic, or heterocyclic and may be substituted, having at least one substituent such as halogen or hydroxyl. Epoxy resin is monomeric or polymeric and having equivalent epoxy weight (EEW) from 150 to 3000 g/eq.

The epoxy resin of step (b) is selected from bisphenol A diglycidyl ether, bisphenol F diglycidyl ether, resorcinol diglycidyl ether, or aliphatic poly functional epoxy resins such as glycol diglycidyl ether, neo-pentyl diglycidyl ether, hexane diglycidyl ether, tri methyl glycidyl ether triglycidyl of trimethalol propane and alike.

The solvent step (c) is selected from xylene, toluene, butanol, methyl isobutyl ketone, phenoxy ethanol, benzyl alcohol, nonyl phenol, 2-hydroxyl ethyl ether of distilled CNSL, dodecanol and higher analogs alone or mixture thereof.

The solvent is used for dilution to reduce viscosity and easy workability with improvement in cured film properties.

In still another embodiment, the present invention provides a process for preparation of polyamide comprising the steps of:
charging soya fatty acid, dimer fatty acid, polyamine preferably diethylene triamine and xylene to afford an Azeotropic mixture and
removing the water of condensation by distillation by raising the temperature of reaction mixture in a range of 200° C. to 250° C. to afford the polyamide product.

The above reaction is as shown in below scheme 3:

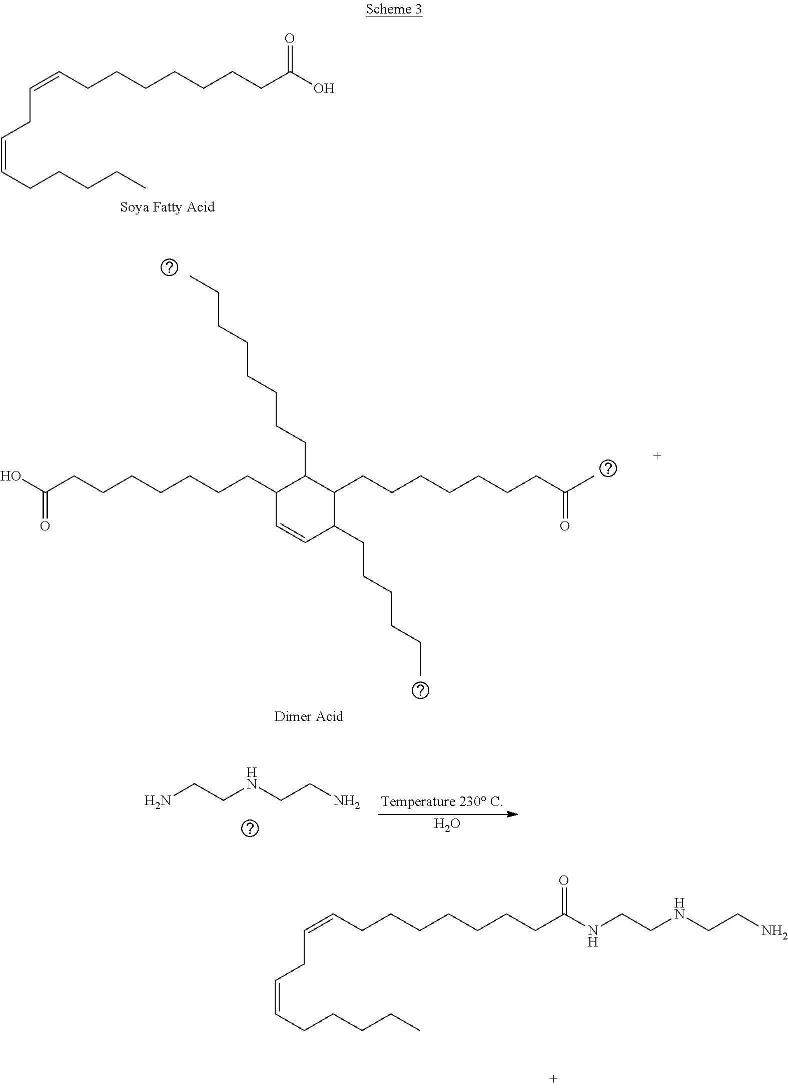 [SMILES: CCCCC/C=C\C/C=C\CCCCCCCC(=O)O ; CCCCCCCCC1C(CCCCCCCC(=O)O)C=CC(CCCCCC)C1CCCCCCCC(C)=O ; NCCNCCN ; O ; CCCCC/C=C\C/C=C\CCCCCCCC(=O)NCCNCCN]

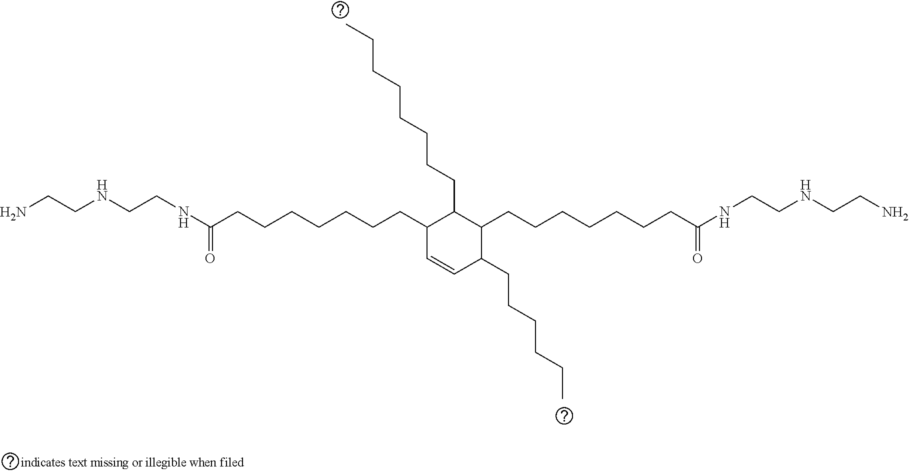 [SMILES: CCCCCCCCC1C(CCCCCCCC(=O)NCCNCCN)C=CC(CCCCCC)C1CCCCCCCC(=O)NCCNCCN]

The fatty acid used for condensation with polyamine to produce polyamide are mono basic or polybasic fatty acids or their mixture. These fatty acids are derived from drying/nondrying oils mainly classified as saturated and unsaturated fatty acids. Some of the commonly used oils to produce fatty acids are Palm, soybean, Rapeseed, Sunflower, Peanut, Cottonseed, Palm kernel, Coconut, Olive, Canola oil, Linseed oil, Rice bran oil, etc. Depending on length of carbon chain separated in short chain (about 5), medium chain (6-12), long chain (13-21) and very long chain (>22) fatty acids. These fatty acids are not single component chemicals they are combination of different acid with different proportion. These fatty acids can be further polymerized by Diels Alder reaction in presence of catalyst to produced higher analog like dimeric, trimeric and polymerized products. The dimeric fatty acid is generally use for synthesis of polyamide but mixture of monomeric or trimeric along with dimer fatty acid can be used as main ingredient.

The polyamine of step (a) is selected from aliphatic, cycloaliphatic, polyoxyalkylene, aminoalkyl, aromatic or alicyclic polyamine alone or mixture thereof.

The aminoalkyl group is preferably an aminomethyl, aminoethyl, aminopropyl or aminobutyl, wherein the alkyl group is either a straight chain or branched. More preferably, the aminoalkyl group is aminomethyl or aminoethyl. Aliphatic, aromatic and alicyclic polyamines can be used solo or in combination with each other depends on targeted final performance properties and end use applications.

The Polyamine of step (a) is selected from Ethylene diamine (EDA), Diethylene triamine (DETA), Triethylenetetramine (TETA), Tetraethylenepentamine (TEPA), Hexamethylenediamine (HMDA) m-Xylenediamine, 1,3-Bis(aminomethyl)cyclohexane, Isophorondiamime (IPD), tetraethylenepentamine (TEPA), pentaethylenehexamine (PEHA), N-aminoethylpiperazine (N-AEP), isophorone diamine (IPDA), 1.3-cyclohexanebis(methylamine) (1,3-BAC); 4,4′-methylenebis(cyclohexylamine) (PACM), xylylenediamine (MXDA), n-aminoethylpiperazine, Menthanediamine alone or mixtures thereof.

In yet another embodiment, the present invention provides a process for the preparation of the Phenalkamine comprising the steps of:
heating the mixture of distilled cashew nut shell liquid i. e. cardanol with polyamine to afford reaction mixture;
adding aldehyde into the reaction mixture of step (a) when temperature of the reaction mixture reached to 70° C. to 80° C. followed by stirring at the same temperature for 3 to 4 hours and removing the water of condensation by raising temperature of reaction mixture to 150° C. to afford the Phenalkamine product.

The aldehyde used in step (a) is selected from paraformaldehyde, acetaldehyde, furfuraldehyde alone or in combination thereof.

The polyamine used in step (a) is selected from aliphatic, aminoalkyl, aromatic or alicyclic polyamine alone or mixture thereof.

The polyamine used in step (a) is selected from Ethylene diamine (EDA), Diethylene triamine (DETA), Triethylenetetramine (TETA), m-Xylenediamine, 1,3-Bis(aminomethyl)cyclohexane, Isophorondiamime (IPD), n-aminoethylpiperazine, Menthanediamine alone or mixtures thereof.

The aminoalkyl group is preferably an aminomethyl, aminoethyl, aminopropyl or aminobutyl, wherein the alkyl group is either a straight chain or branched. More preferably, the aminoalkyl group is aminomethyl or aminoethyl.

Aliphatic, aromatic and alicyclic polyamines can be used solo or in combination with each other depends on targeted final performance properties and end use applications.

The Phenalkamine is selected from Phenalkamine A or Phenalkamine B.

The mole ratio of polyamine to cardanol is within the range of 1:1 to 5:1, more preferably from about 1:0.8 to about 2:1.

The mole ratio of the polyamine to aldehyde compound is within the range of 1:1 to 1:4, preferably about 1:2 to about 1:3.

On an equivalents basis, the ratio of aldehyde and amine should be more than or equal to one mole of amine per equivalent of the phenolic compound.

The cardanol is distilled product of CNSL with content at least 80 weight % to 100 weight % of the cashew nutshell liquid, based on a total weight of component. The distilled product of CNSL mainly includes cardanol as a primary component and additionally include cardol, methylcardol, as secondary components. The composition of cardanol varies based on degree of unsaturation inside chain. Cardanol is mixture of tri-unsaturated cardanol (41%) which is major component, 34% mono-unsaturated, 22% bi-unsaturated, and 2% saturated.

The synthesis of Phenalkamine A and Phenalkamine B are shown in following schemes 4 and 5.

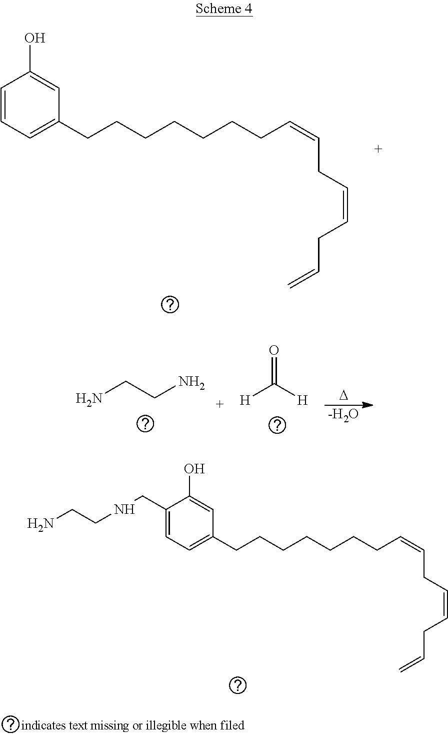

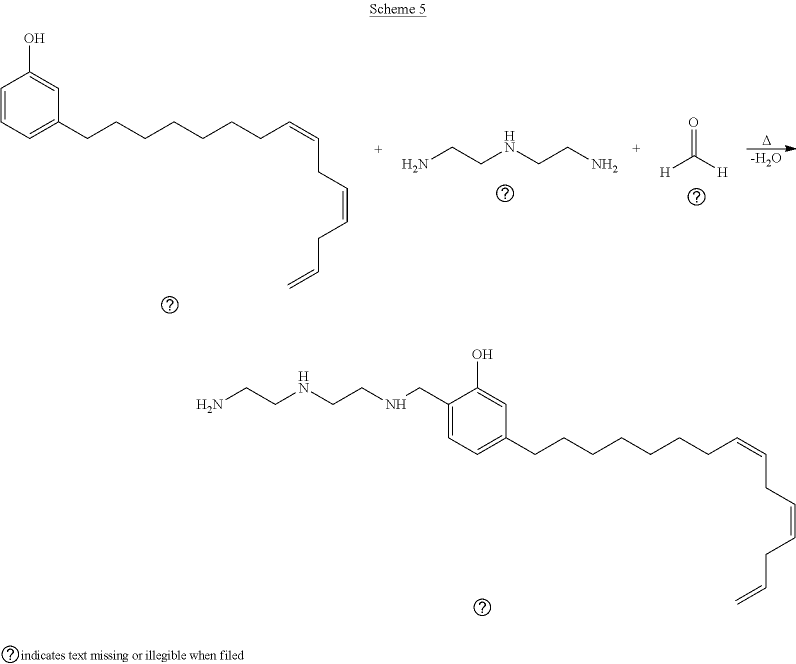

In still yet another embodiment, the present invention provides a coating composition comprising:
a) the phenalkamide curing agent of general formula (I);
b) an epoxy resin having on average more than one glycidyl group per molecule
and c) pigments

The epoxy resin is selected from bisphenol A diglycidyl ether, bisphenol F diglycidyl ether, resorcinol diglycidyl ether, or aliphatic poly functional epoxy resins such as glycol diglycidyl ether, neo-pentyl diglycidyl ether, hexane diglycidyl ether, tri methyl glycidyl ether triglycidyl of trimethalol propane, alone or combination thereof.

The coating composition shows excellent in workability, have good pot life, shelf-life stability, drying characteristics, corrosion resistance, and adhesion when compared with Phenalkamine and polyamide systems.

### EXAMPLES

Following examples are given by way of illustration therefore should not be construed to limit the scope of the invention.

### Example 1: Synthesis of Polyamide Intermediate

A Polyamide is produced by condensation reaction of fatty acid with aliphatic polyamine. In particular, 50 gm of soya fatty acid, 20 gm dimer fatty acid, 27 gm of Diethylene Triamine and 3 gm of xylene to form Azeotropic mixture are charged in four-necked round bottom flask equipped with a condenser, thermometer, a mechanical stirrer, and a nitrogen connection to form a reaction mixture. Then, flask is thoroughly purged and protected with nitrogen, agitation of the reaction mixture within the flask is started and heat is applied to the reaction mixture. After that, water of condensation was removed by Azeotropic distillation by raising temperature of reaction mixture to 230° C. The product obtained with color 8, Acid value 1.2 mg KOH/gm, Amine value 290 mg/KOH, and viscosity @ 25° C. was 500 cps.

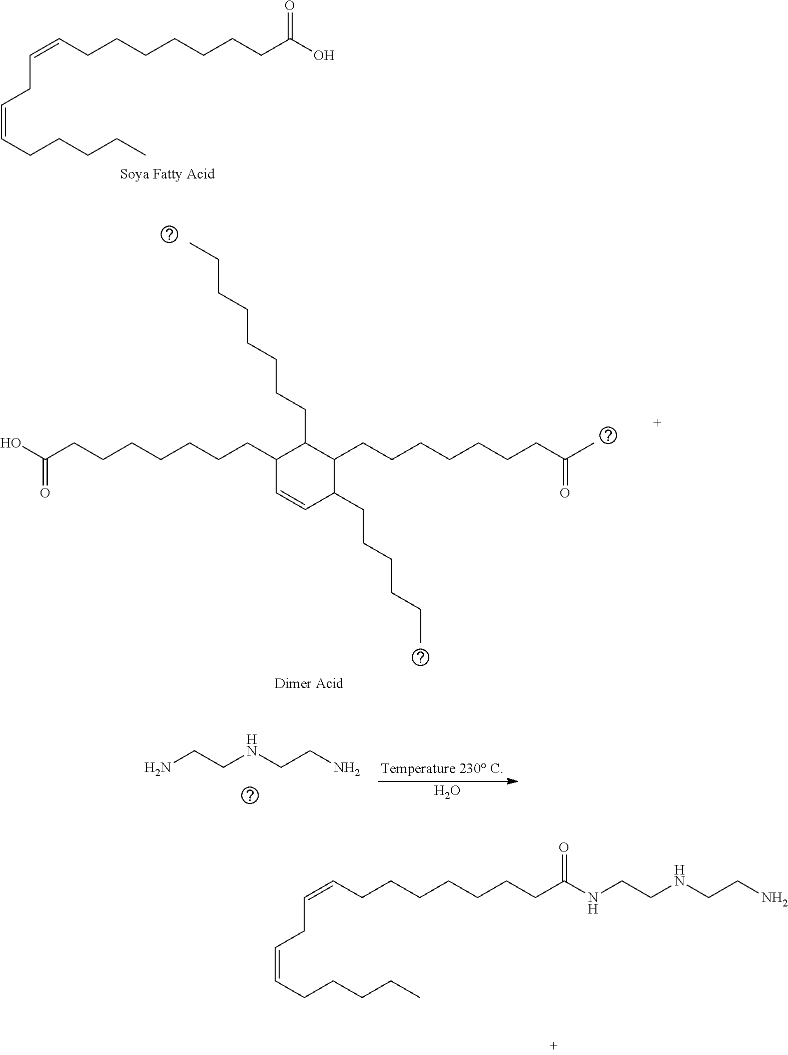 [SMILES: CCCCC/C=C\C/C=C\CCCCCCCC(=O)O ; CCCCCCCCC1C(CCCCCCCC(=O)O)C=CC(CCCCCC)C1CCCCCCCC(C)=O ; NCCNCCN ; O ; CCCCC/C=C\C/C=C\CCCCCCCC(=O)NCCNCCN]

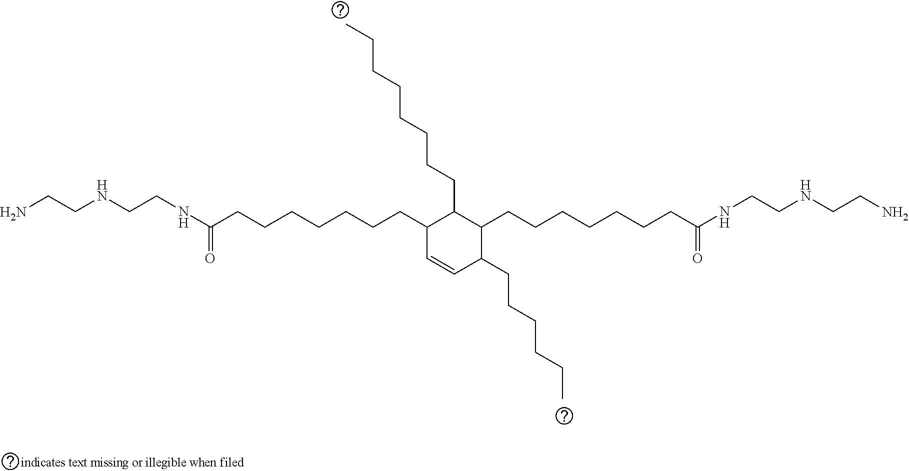 [SMILES: CCCCCCCCC1C(CCCCCCCC(=O)NCCNCCN)C=CC(CCCCCC)C1CCCCCCCC(=O)NCCNCCN]

### Example 2: Synthesis of Phenalkamine A

Phenalkamine A is produced by reaction of distilled cashew nut shell liquid i.e. cardanol with aliphatic polyamine and paraformaldehyde. In particular, 70 gm of cardanol, 16 gm Ethylene Diamine are charged in four- necked round bottom flask equipped with a condenser, thermometer, a mechanical stirrer, and a nitrogen connection to form a reaction mixture. Then, the flask is thoroughly purged and protected with nitrogen, agitation of the reaction mixture within the flask is started and heat is applied to the reaction mixture. Once a temperature of 70° C. is reached, 14 gm of paraformaldehyde was added in reaction mixture. Exotherm is control by cooling reaction mixture and temperature is maintained between 70 to 80° C. for 3-4 hr. After that, water of condensation was removed by raising temperature of reaction mixture to 150° C. The product obtained with color 14, amine value 310 mg/KOH, and viscosity @ 25° C. was 30000 cps.

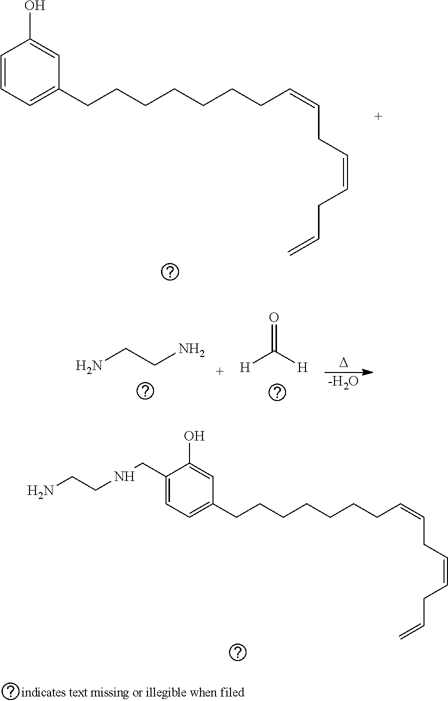

### Example 3: Synthesis of Phenalkamine B

Phenalkamine B is produced by reaction of distilled cashew nut shell liquid i. e. cardanol with aliphatic polyamine and paraformaldehyde. In particular, 69 gm of cardanol, 20 gm Diethylene triamine are charged in four- necked round bottom flask equipped with a condenser, thermometer, a mechanical stirrer, and a nitrogen connection to form a reaction mixture. Then, the flask is thoroughly purged and protected with nitrogen, agitation of the reaction mixture within the flask is started and heat is applied to the reaction mixture. Once a temperature of 70° C. is reached, 11 gm of paraformaldehyde was added in reaction mixture. Exotherm is control by cooling reaction mixture and temperature is maintained between 70 to 80° C. for 3-4 hr. After that, water of condensation was removed by raising temperature of reaction mixture to 150° C. The product obtained with color 14, amine value 290 mg/KOH.

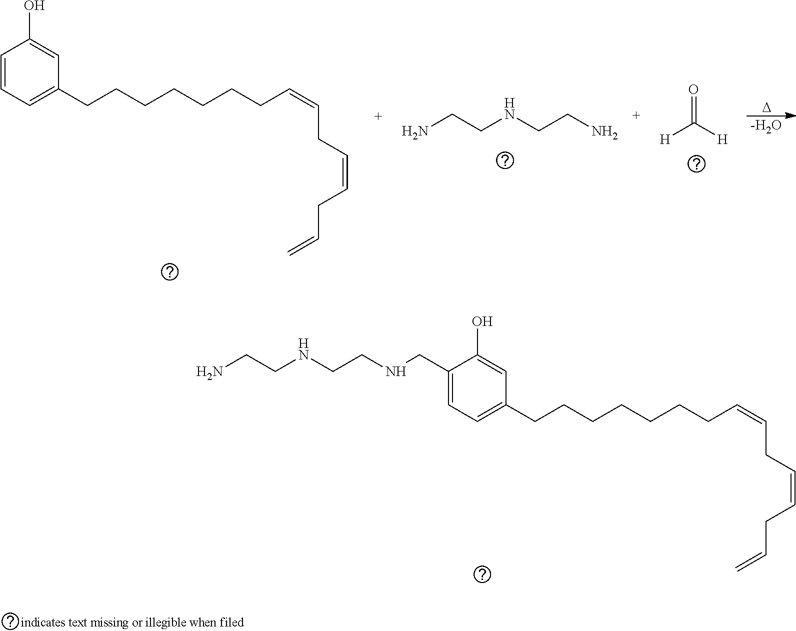

### Example 4: Synthesis of Phenalkamide PA-1

The Polyamide obtained in example 1 is combined with phenalkamine A with liquid epoxy resin EEW 190, solution in inert solvent were produced to give final phenalkamide curing agent. In particular, 6 gm of product from Example 1, 35 gm of phenalkamine A is charged in four-necked round bottom flask equipped with a condenser, thermometer, a mechanical stirrer, and a nitrogen connection to form a reaction mixture for producing phenalkamine. Under agitation heat is applied to the reaction mixture. Once a temperature of 70-80° C. is reached, add 9 gm of liquid epoxy resin with EEW 190 over period of 1 hr. After addition maintain temperature for 2-3 hr, nitrogen protection is continued until the formation of phenalkamide. The reaction mixture is cooled to 50° C. and final phenalkamide is produce by addition of 40 gm of xylene, and 10 gm butanol. The phenalkamine was produce with Color 14, Amine value 140 mg/KOH, Solid content 50%, and Viscosity @ 25° C. was 60 cps.

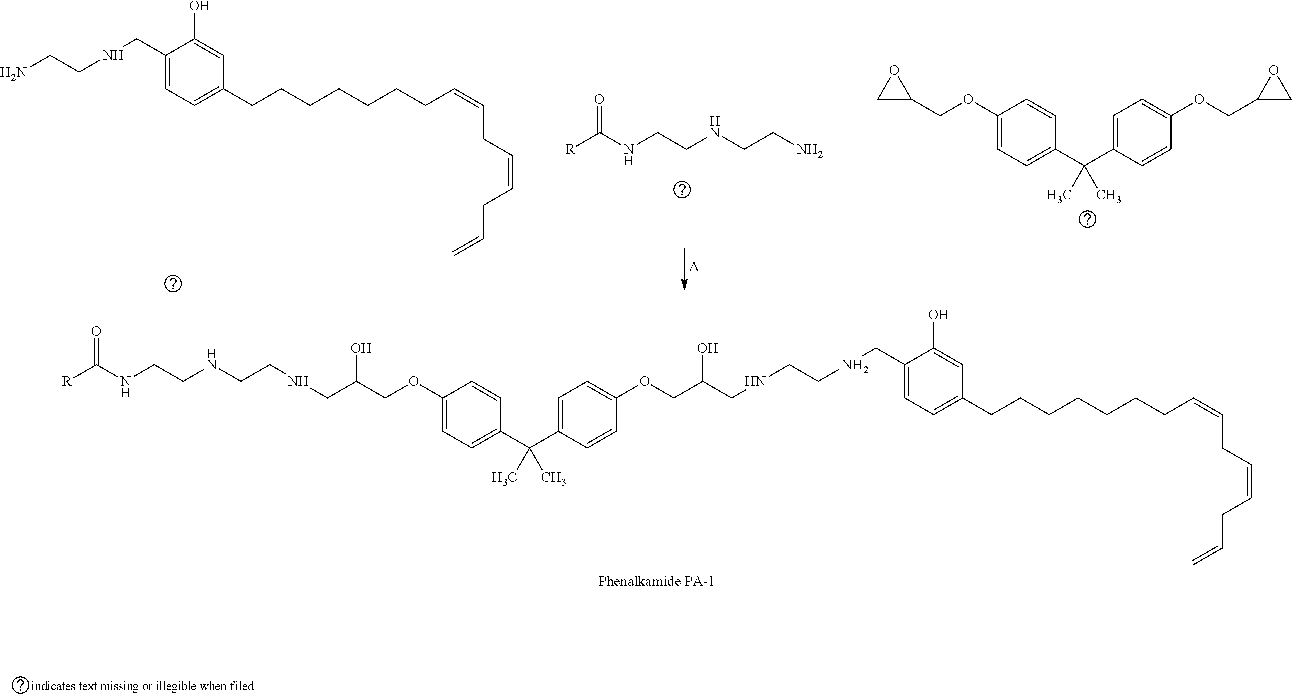

### Example 5: Synthesis of Phenalkamide PA-2

Phenalkamide PA-2 is produced with polyamide resin from example 1 and phenalkamine B. In particular, 13 gm of product from Example 1, and 70 gm of phenalkamine B is charged in four-necked round bottom flask equipped with a condenser, thermometer, a mechanical stirrer, and a nitrogen connection to form a reaction mixture for producing phenalkamine. Under agitation heat is applied to the reaction mixture. Once a temperature of 70-80° C. is reached, add 8 gm of liquid epoxy resin with EEW 190 over period of 1 hr. After addition maintain temperature for 2-3 hr, nitrogen protection is continued until the formation of phenalkamide. The reaction mixture is cooled to 50° C. and final phenalkamide is produce by addition of 9 gm of 2-hydroxyl ethyl ether of distilled CNSL. The phenalkamine was produce with color 15, Amine value 265 mg/KOH, and Viscosity 75,000 cps @ 25° C.

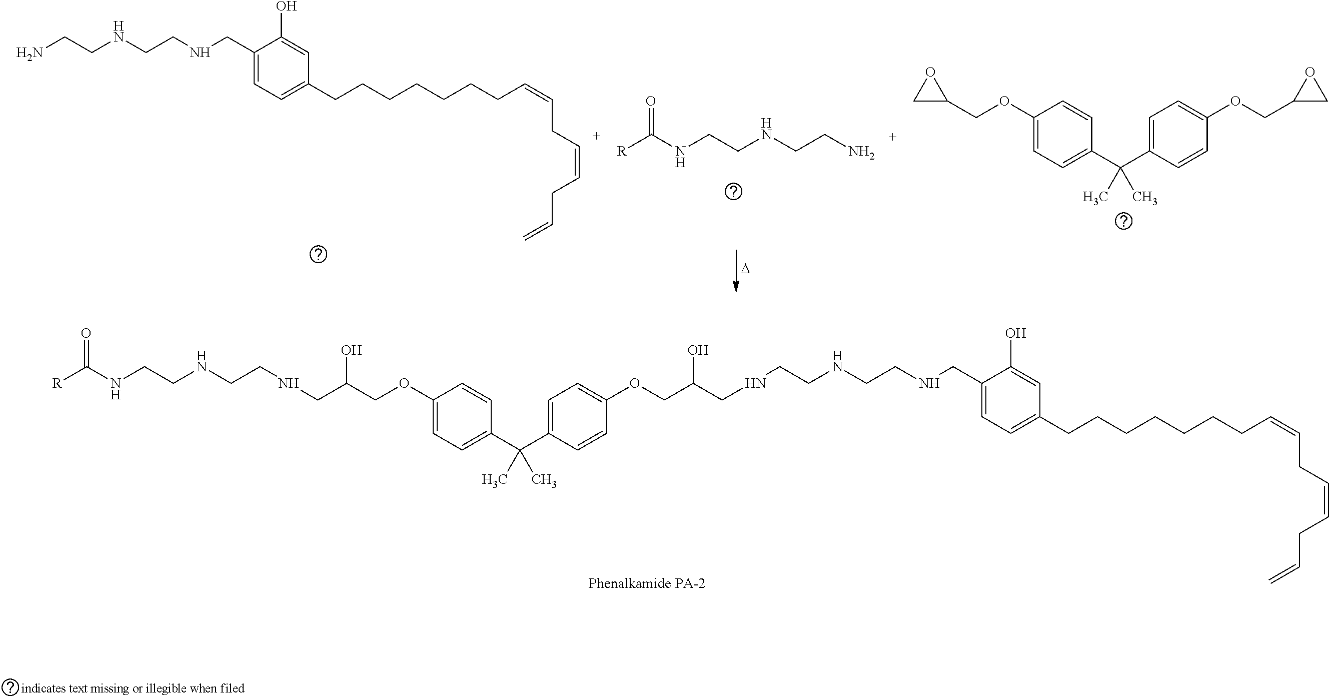

### Example 6: 2K Epoxy Paint Composition

Epoxy paint composition is divided in two parts, Part A Base with epoxy resin and part B containing epoxy curing agent. All of the Base ingredients were combined in the High Speed dissolver. The ingredients of Part A were combined according to the order reflected in Table 1. Once all of the ingredients were combined, the ingredients were Mix in a high speed dissolver until a smooth finish on panel was achieved. Once this smooth finish was achieved, the temperature of the blend was brought to approximately 120° F. (approximately 48.9° C.) and held for approximately 20 minutes while the ingredients were continuously agitated.

Part B is Epoxy curing agent reflected in Table 1, was kept separate. At the time of coating application Mix. the Base & Hardener part as per weight reflected in table 1.

TABLE 1
Coating Paint Compositions

| Description | Example 4 | Example 5 | Example 6 | Example 7 |
|---|---|---|---|---|
| Part -A | | | | |
| ¹Epoxy Resin | 18.58 | 20.33 | 20.33 | 21.50 |
| ( Ep-Eq.-480 ) | | | | |
| ²Nuosper-657 | 0.30 | 0.30 | 0.30 | 0.30 |
| Xylene | 19.92 | 18.17 | 18.17 | 17.00 |
| Butanol | 3.00 | 3.00 | 3.00 | 3.00 |
| ³Titanium Di-oxide | 3.00 | 3.00 | 3.00 | 3.00 |
| ⁴Zinc Phosphate | 3.00 | 3.00 | 3.00 | 3.00 |
| ⁵Silica ( 20 micron) | 12.00 | 12.00 | 12.00 | 12.00 |
| ⁷Steatite (20 micron) | 12.00 | 12.00 | 12.00 | 12.00 |
| ⁸Marble Powder | 12.00 | 12.00 | 12.00 | 12.00 |
| (20 micron) | | | | |
| ⁹Namlon T-206 | 0.500 | 0.500 | 0.500 | 0.500 |
| ¹⁰Tinter Black | 0.400 | 0.400 | 0.400 | 0.400 |
| Part-B | | | | |
| ¹¹Polyamide-125 | — | — | 5.09 | — |
| ¹²PPA-7041 | — | — | — | 4.21 |
| Phenalkamide PA-2 | — | 5.09 | — | — |
| Phenalkamide PA-1 | 6.39 | — | — | — |
| Xylene | 8.91 | 10.21 | 10.21 | 11.09 |

¹commercially available from Huntsman
²commercially available from ELEMENTIS SPECIALTIES (INDIA) PRIVATE LIMITED
³commercially available from Dupont
^{5,6,7,8}commercially available from 20 Microns Ltd.
⁹commercially available from Kusumoto Chemicals Ltd.
¹¹commercially available from Air Products.
¹²commercially available from Paladin Paints & Chemicals (Pvt.) Ltd.

### Example 7: Paint Composition of Epoxy and Polyurethane (Two Pack) for Over Coating

Coating Compositions was prepared for over coating using the components and amounts identified in Table 2. All of the ingredients were combined in the Mill. In Examples, the ingredients of Part A were combined according to the order reflected in Table 2. Once all of the ingredients were combined, the ingredients were grind at Mill until a Hegman reading greater than 7.00 was achieved. Once this Hegman reading was achieved, the temperature of the blend was brought to approximately 120° F. (approximately 48.9° C.) and held for approximately 20 minutes while the ingredients were continuously agitated. Next, Part B was prepared by combining the ingredients under agitation in the order reflected in Table. Part B was added slowly to grinding machine for flushing after that, add the flush material into Part A under agitation. Part C was prepared by combining the ingredients under agitation in the order reflected in Table 2. Part C was added slowly to Part A under agitation.

Part D was then prepared by combining the ingredients reflected in Table 2 under agitation. Then, Part D was added under agitation at the time of application to the already combined blend of Parts A, B & C.

TABLE 2
2K Epoxy and Polyurethane Paint composition for over coating

| Description | Example 8 | Example 9 |
|---|---|---|
| Part-A | | |
| ¹Epoxy Resin ( Ep-Eq.-480 ) | 12.00 | — |
| ¹⁵Acrylic Polyol ( OH-Value 50 ) | — | 12.00 |
| ¹⁶Urea Formaldehyde Resin | 1.00 | — |
| Nuosper-657 | 0.30 | 0.30 |
| Ethyl Cello solve Acetate | — | 1.5 |
| Xylene | 8.00 | 8.00 |
| Butanol | 1.5 | — |
| MIBK | 1.5 | — |
| Titaniμm Di-oxide | 16.00 | 19.00 |
| ¹⁵Bent one Jelly 10% in Xylene. | 7.00 | 2.00 |
| Part-B | | |
| Xylene | 4.00 | 1.5 |
| Butanol | 1.50 | — |
| Ethyl Cello solve Acetate | — | 1.5 |
| Part-C | | |
| Epoxy Resin ( Ep-Eq.-480 ) | 33.84 | — |
| Xylene | 4.02 | — |
| MIBK | 1.50 | — |
| ¹⁵Acrylic Polyol (OH-Value 50) | — | 43.00 |
| Ethyl Cello solve Acetate | — | 1.20 |
| Part-D | | |
| ¹¹Polyamide-125 | 7.84 | — |
| ¹⁷Aliphatic Isocyanate | — | 7.84 |
| Butyl Acetate | — | 2.16 |

¹⁵commercially available from Nuplex Polymers.
¹⁶commercially available from Synpol,
¹⁷commercially available from Bayer Material Science,

The test results for coatings prepared according to formulations indicated in table 2, in comparison with phenalkamine and polyamide systems are presented in Tables 4.

### Example 8 (Two Pack): Paint Composition of Epoxy Finish Paint for Outdoor Exposure Test

All of the ingredients were combined in the Mill. In Examples, the ingredients of Part A were combined according to the order reflected in table 3. Once all of the ingredients were combined, the ingredients were grind at Mill until a Hegman reading greater than 7.00 was achieved. Once this Hegman reading was achieved, the temperature of the blend was brought to approximately 120° F. (approximately 48.9° C.) and held for approximately 20 minutes while the ingredients were continuously agitated. Next, Part B was prepared by combining the ingredients under agitation in the order reflected in Table. 3, Part B was added slowly to grinding machine for flushing after that, add the flush material into Part A under agitation. Part C was prepared by combining the ingredients under agitation in the order reflected in Table 3. Part C was added slowly to Part A under agitation.

Part D was then prepared by combining the ingredients reflected in Table 3 under agitation. Then, Part D was added under agitation at the time of application to the already combined blend of Parts A, B & C.

TABLE 3
Paint composition for outdoor Exposure performance test

| Description | Example-10 | Example-11 | Example-12 | Example-13 |
|---|---|---|---|---|
| Part-A (Base) | | | | |
| Epoxy Resin | 12.00 | 12.00 | 12.00 | 12.00 |
| ( Ep-Eq.-480 ) | | | | |
| Urea | 1.00 | 1.00 | 1.00 | 1.00 |
| Formaldehyde | | | | |
| Resin | | | | |
| Nuosper-657 | 0.30 | 0.30 | 0.30 | 0.30 |
| Xylene | 8.00 | 8.00 | 8.00 | 7.55 |
| Butanol | 1.5 | 1.50 | 1.5 | 1.50 |
| MIBK | 1.5 | 1.50 | 1.5 | 1.50 |
| Titanium | 16.00 | 16.00 | 16.00 | 16.00 |
| Di-oxide | | | | |
| Bentone Jelly | 7.00 | 7.00 | 7.00 | 7.00 |
| 10% in Xylene. | | | | |
| Part-B | | | | |
| Xylene | 4.00 | 4.00 | 4.00 | 2.50 |
| ⁵Butanol | 1.50 | 1.50 | 1.50 | 1.50 |
| Part-C | | | | |
| Epoxy Resin | 26.60 | 30.22 | 30.22 | 32.65 |
| ( Ep-Eq.-480 ) | | | | |
| Xylene | 5.83 | 2.21 | 2.21 | 1.00 |
| MIBK | 1.50 | 1.50 | 1.50 | 1.50 |
| Part-D | | | | |
| Polyamide-125 | — | — | 10.55 | — |
| Phenalkamide | 13.27 | | — | — |
| PA-1 | | | | |
| Phenalkamide | — | 10.55 | — | — |
| PA-2 | | | | |
| PPA-7041 | — | — | — | 8.72 |
| Xylene | 0.73 | 3.45 | 3.45 | 5.28 |

The phenalkamide systems synthesized was tested for outdoor exposure performance compared against polyamide and phenalkamine chemistries. Exterior exposure test results are reported in table 5. Test results shows, hybrid phenalkamide systems has intermediate outdoor stability compared to phenalkamine and polyamide which is superior than phenalkamine.

TABLE 4
Physical & Chemical properties paint composition

| | Example 4 | Example 5 | Example 6 | Example 7 |
|---|---|---|---|---|
| Viscosity on Ford | 52 Secs. | 31 Secs. | 46 Secs. | 31 Secs. |
| Cup B4 @30° C. | | | | |
| B.K. Drying | | | | |
| Time @ 5° C. | | | | |
| Touch Dry | 30 Min. | 30 Min. | 1 hr | 30 Min. |
| Hard Dry | 8 Hrs. | 9 Hrs. | >24 Hrs | 10 Hrs. |
| Drying Time | | | | |
| AT 30° C. | | | | |
| Touch Dry | 30 Min | 30 Min | 1 hr | 15 Min.. |
| Hard Dry | 4 hr | 5 hr | 8 hr | 4 hr. |
| Solid content at | 68 ± 2 | 68 ± 2 | 68 ± 2 | 68 ± 2 |
| 120° C./1 hr | | | | |
| Density as per | 1.31 ± 0.04 | 1.31 ± 0.04 | 1.31 ± 0.04 | 1.31 ± 0.04 |
| ASTM D-1475-98 | | | | |
| Performance | | | | |
| properties | | | | |
| Adhesion On Mild | A | A | A | A |
| steel surface. | | | | |
| Impact Resist.as | A | A | A | A |
| per ASTM-D-2794- | | | | |
| 93.Direct 15″ | | | | |
| Flexibility as per | A | A | A | A |
| ASTM-D-522-93a. | | | | |
| Corrosion Resistance | A | A | B | A |
| as per ASTM-B- | | | | |
| 117 at 4O to 45 μm | | | | |
| DFT on Mild | | | | |
| Steel surface, after | | | | |
| 240 Hrs. | | | | |
| Over Coating | | | | |
| Adhesion after 24 hr | | | | |
| Example 8 | A | A | A | A |
| system @ 40 μm | | | | |
| Example 9 | A | A | A | A |
| system @ 40 μm | | | | |
| Over Coating | | | | |
| Adhesion after | | | | |
| 3 months | | | | |
| Example 8 | A | A | A | C |
| system @ 40 μm | | | | |
| Example 9 | A | A | A | C |
| system @ 40 μm | | | | |
| Pot Life @ 30° C. | 9 hr | 10 hr | 12 Hrs. | 8 hr |
| ( 100 gm.) | | | | |

TABLE 5
Comparative Exterior Exposure Performance.

| | Example 10 | | Example 11 | | Example 12 | | Example 13 | |
|---|---|---|---|---|---|---|---|---|
| | | Gloss | | Gloss | | Gloss | | Gloss |
| | Visual | Retention | Visual | Retention | Visual | Retention | Visual | Retention |
| After 2 | B | 70% | B | 70% | B | 80% | C | 60% |
| Months | | | | | | | | |
| After 4 | C | 55% | C | 55% | B | 75% | D | 40% |
| Months | | | | | | | | |
| After 6 | D | 40% | D | 40% | C | 50% | D | 30% |
| Months | | | | | | | | |

### Example 9: Test Methods

A metal plate measuring 0.8×100×150 mm is Sand by 400 emery paper and after that Spray coated with the 2K epoxy coating composition to a dry film thickness of about 45 μm, and the coating is dried at 25° C. and 65% RH for 7 days giving a test specimen.

Unless indicated otherwise, the following test methods were utilized in the Examples that follow.

Acid Value

Acid value is evaluated according to ASTM D 1980 titration method, indicated as mg KOH/gm.

Amine Value

Amine value of phenalkamine produced was tested according to ASTM D2074. The indictor titration method was used and value of test are mentioned as mg/KOH.

Viscosity

Viscosity is measured according to ASTM D 2196 with Brookfield digital viscometer LVDV model having multiple rotational speed. When not mentioned parameters are spindle number 63, rotation speed 30 RPM and temperature 25° C. for testing.

Color

Gardner scale color of product is mentioned following ASTM D1544. Color of the test specimen was compared with standard Gardner scale and close match is mentioned.

Solid Content

Solid content mentioned was tested following ASTM D1259. Volatile matter present in sample was evaporated by heating in oven for 1 hr. and solid content is calculated as percentage comparing weight of sample before and after heating.

Drying Performance

Phenalkamine produced are cross linked with liquid epoxy resin EEW 190 to test performance properties of cured film. ASTM D 5895 was followed to record test results. Drying performance are tested at 25° C. and 5° C. with 200 μm DFT on B. K. Drying recorder

Adhesion Test

Adhesion testing was performed to assess whether the coating compositions adhere to the coated substrate. The Adhesion Test was performed according to ASTM D 3359 -Test method.

The results are rated as follows:
A: No abnormalities,
B: Peelings on part of the coating surface,
C: Peelings on the entire coating surface

Impact Resistance.

This test method covers a procedure for rapidly deforming by impact a coating film and its substrate and for evaluating the effect of such deformation test was performed according to ASTM-D-2794-93.

The results are rated as follows:
A: No abnormalities,
B: Peelings on part of the coating surface,
C: Peelings on the entire coating surface

Flexibility

Testing was performed to determine whether the coating compositions resistance to cracking to the coated substrate, test was performed according to ASTM-D-522-93a.

The results are rated as follows:
A: No abnormalities,
B: Peelings on part of the coating surface,
C: Peelings on the entire coating surface

Corrosion Resistance

Panel Preparation Two Mild steel panels of size 100×150 mm which is sand by 400 No. emery paper were taken for paint application, Apply Paint by Spray application, DFT of the applied coating is 45 μm. After 7 days of ageing sealed the edges with Adhesive tape and Expose for the testing.

The corrosion resistance was check with standard method ASTM B117. The test panel were prepared and subjected to salt spray conditions and evaluated for rusting and blisters. The condition of the coating Surface was rated as follows:
A: No abnormalities,
B: Partially rusted or blistered,
C: Entirely rusted or blistered

Exterior Exposure Test.

Exterior exposure test was performed according to ASTM D-1014-02. Two Mild steel panels of size 100×150 mm which is sand by 400 No. polish paper were taken for paint application, apply Two coats each of 45 μm2K Epoxy White Paint composition with Polyamide, Phenalkamide, Phenalkamine curing agents. After 7 days of curing sealed the edges with Adhesive tape and use for outdoor exposure testing. Similarly, prepared control sample for comparatives study. Expose the panel at the angle of 45° facing to south equator for 12 months. Exposed panels were tested after each 2 months visually for Rusting, Checking, Cracking, Blistering, flaking also for gloss retention against controlled sample and Test reports are mention as average of duplicate samples.

Surface was rated as follows for visual inspection.
A: No abnormalities,
B: Slightly Yellowing, No Rusting, Checking, Cracking, Blistering.
C: Yellowing Partially Rusting, Checking, Cracking, Blistering or Flaking.
D: Total Shade change, Entirely Rusting, Checking, Cracking, Blistering or Flaking.

The results of this test for coatings prepared according to the present invention are presented in Table 5.

We claim:

1. A phenalkamide curing agent of general formula (I) represented as: 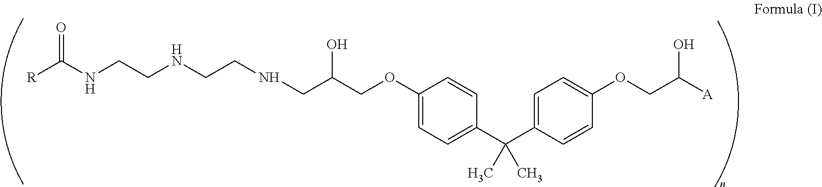
wherein,
R is selected from hydrogen, or (un)substituted or substituted hydrogen, alkyl, alkenyl or alkynyl; or (un)substituted or substituted aryl, heteroalkyl, heteroaryl, arylalkyl, heteroarylalkyl; (un) substituted or substituted cycloalkyls, cycloalkenyl or cycloalkynyl; azo, amino, halo, nitro, cyano, hydroxyl, carbonyl, thiocarbonyl, carboxylic, alkoxy, carbamide, carbamate, hydrazine, sulfonyl, sulphide, thioether, sulphonamide, phosphates;
A is selected from (un)substituted or substituted alkyl, alkenyl or alkynyl; or (un)substituted or substituted aryl, heteroalkyl, heteroaryl, arylalkyl, heteroarylalkyl; (un) substituted or substituted cycloalkyls, cycloalkenyl or cycloalkynyl; azo, amino, halo, nitro, cyano, hydroxyl, carbonyl, thiocarbonyl, carboxylic, alkoxy, carbamide, carbamate, hydrazine, sulfonyl, sulphide, thioether, sulphonamide, phosphates;
n is selected from 1 to 4.

2. The phenalkamide curing agent of general formula (I) as claimed in claim 1 comprising:

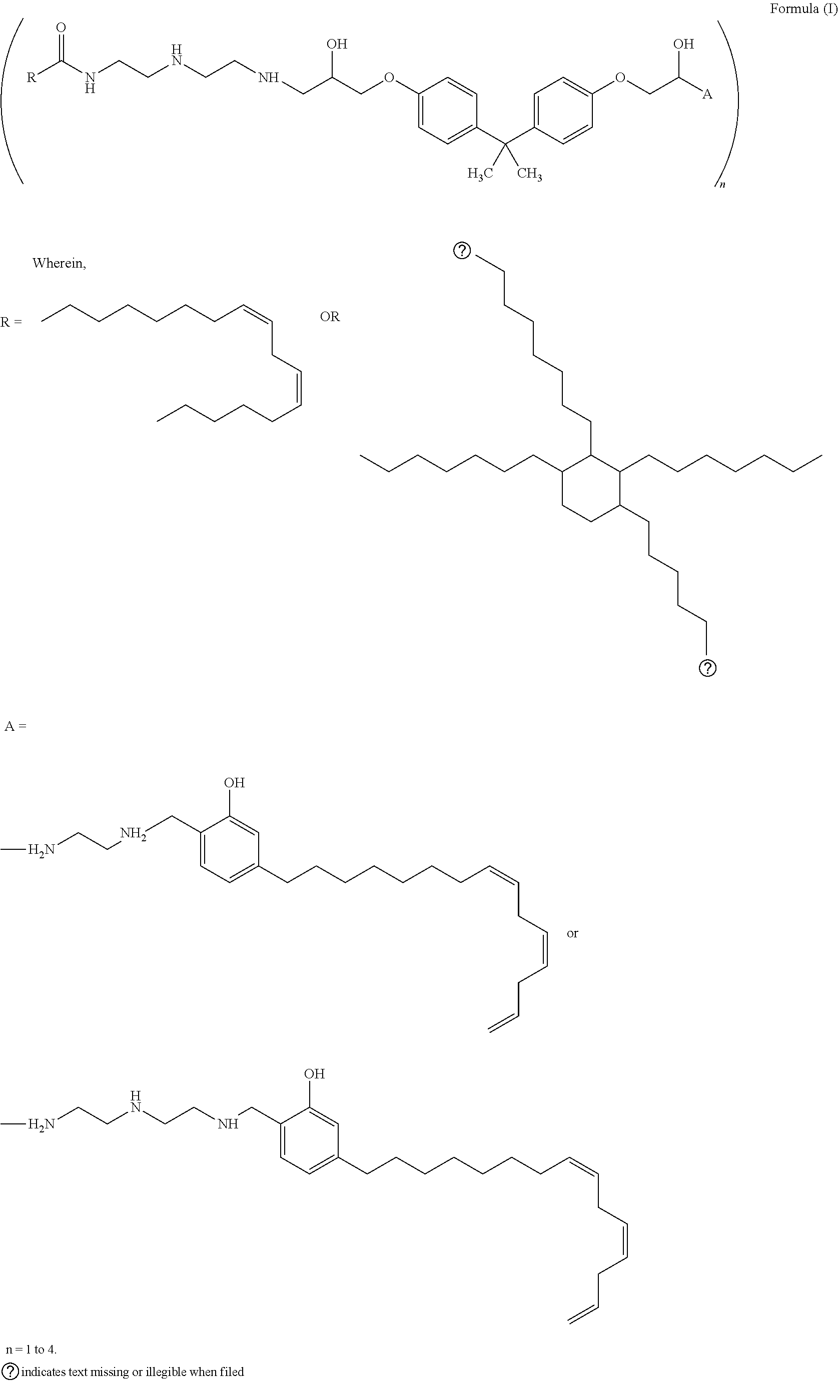

3. The phenalkamide curing agent of formula (I) as claimed in claim 1, wherein said curing agent is selected from

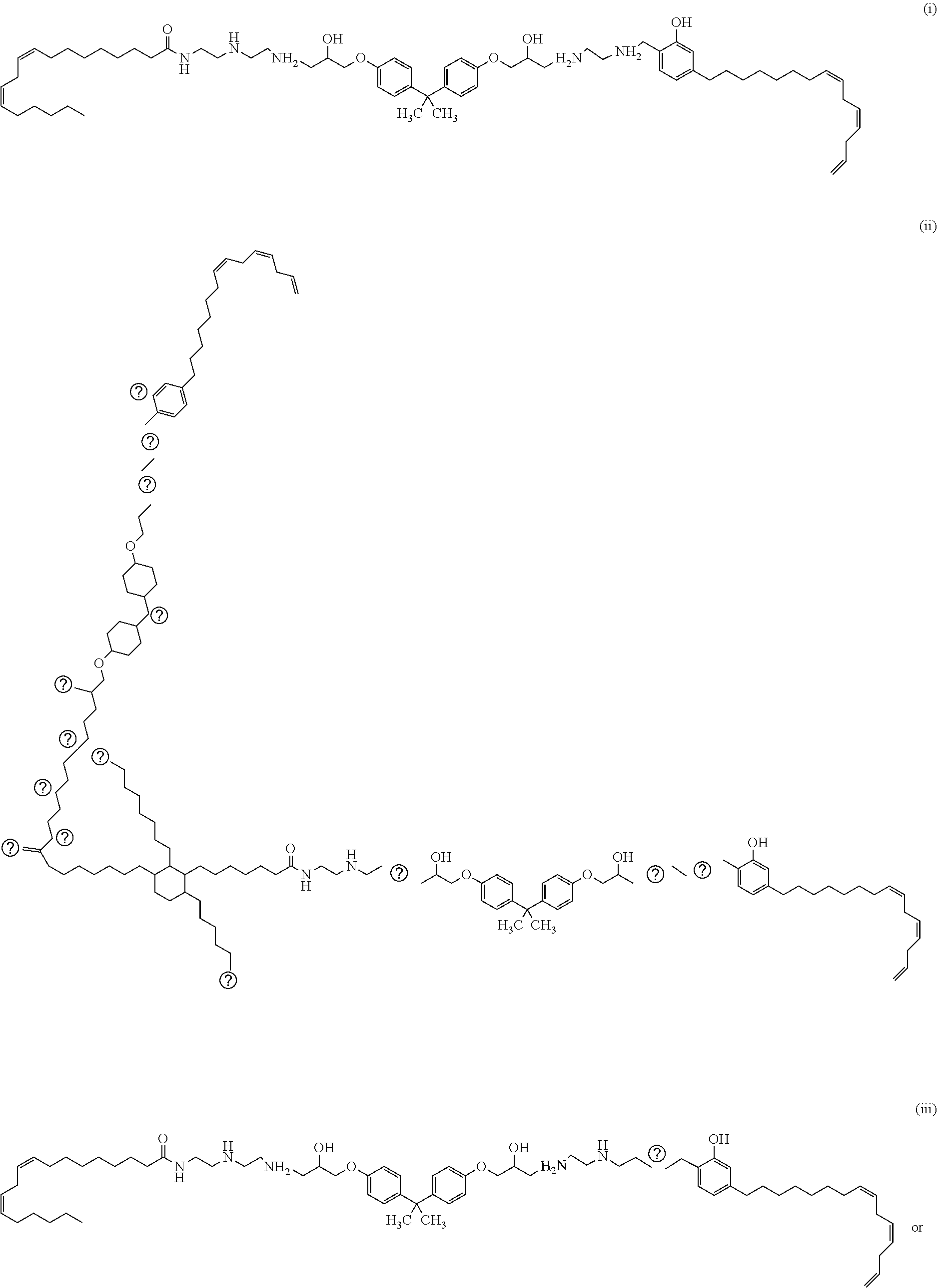

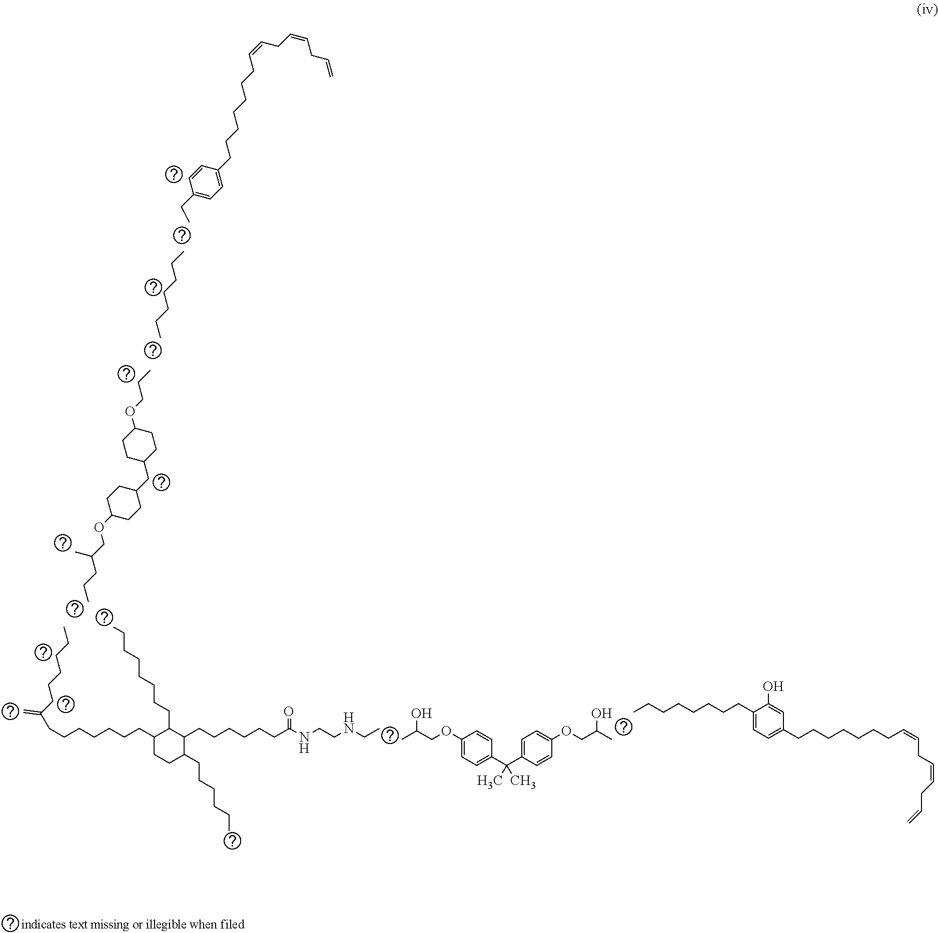

4. A process for preparation of the phenalkamide curing agent of general formula (I) as claimed in claim 1 comprising the steps of:
a) charging a mixture of phenalkamine A or B and polyamide to obtain a reaction mixture;
b) adding epoxy resin into the reaction mixture of step (a) over a period of 1 to 2 hours when temperature of the reaction mixture reaches at a temperature range 70° C. to 80° C. and followed by the stirring at the same temperature for 2 to 3 hours; and
c) cooling the reaction mixture of step (b) upto 40° C. to 50° C. and adding the inert solvent to reduce the viscosity to afford the phenalkamide curing agent of general formula (I).

5. The process as claimed in claim 4, wherein said phenalkamine A or B of step (a) are represented as below:

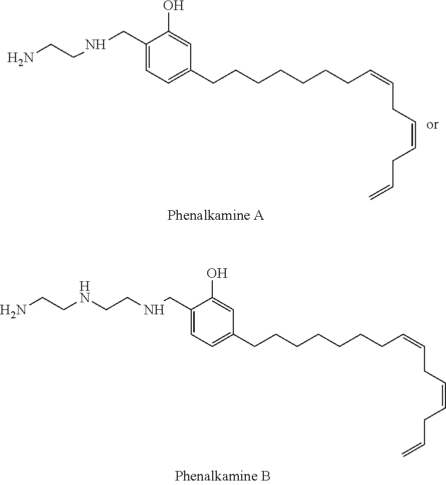 [SMILES: C=CC/C=C\C/C=C\CCCCCCCc1ccc(CNCCN)c(O)c1 ; C=CC/C=C\C/C=C\CCCCCCCc1ccc(CNCCNCCN)c(O)c1]

6. The process as claimed in claim 4, wherein said polyamide of step (a) is represented as below: 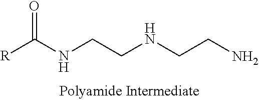
R is selected from

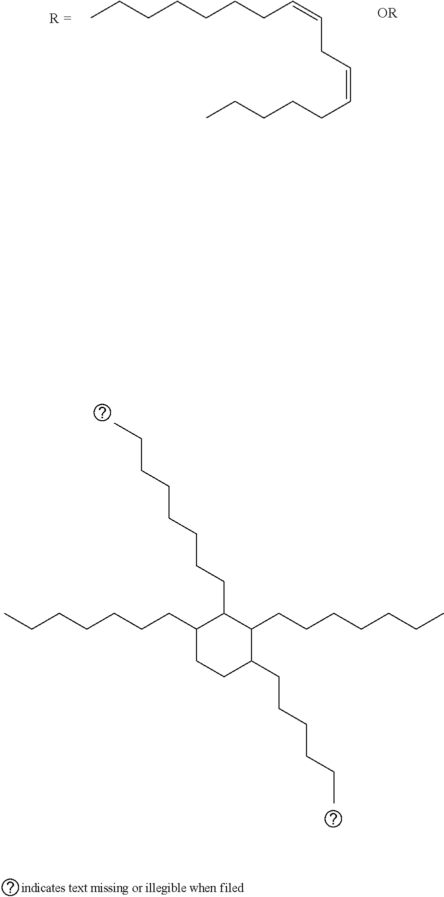

7. The process as claimed in claim 4, wherein said epoxy resin of step (b) is monomeric or polymeric and having equivalent epoxy weight (EEW) from 150 to 3000 g/eq.

8. The process as claimed in claim 4, wherein said epoxy resin of step (b) is selected from bisphenol A diglycidyl ether, bisphenol F diglycidyl ether, resorcinol diglycidyl ether, or aliphatic poly functional epoxy resins selected from glycol diglycidyl ether, neo-pentyl diglycidyl ether, hexane diglycidyl ether, tri methyl glycidyl ether triglycidyl of trimethalol propane and alike.

9. The process as claimed in claim 4, wherein said solvent step (c) is selected from xylene, toluene, butanol, methyl isobutyl ketone, phenoxy ethanol, benzyl alcohol, nonyl phenol, 2-hydroxyl ethyl ether of distilled CNSL, dodecanol and higher analogs alone or mixture thereof

10. The process as claimed in claim 4, wherein said polyamide is prepared by process comprising the steps of:
a) charging soya fatty acid, dimer fatty acid, polyamine preferably diethylene triamine and xylene to afford azetropicmixture; and
b) removing water of condensation by distillation by raising the temperature of reaction mixture in a range of 200° C. to 250° C. to afford polyamide product.

11. The process as claimed in claim 10, wherein said Polyamine of step (a) is selected from Ethylene diamine (EDA), Diethylene triamine (DETA), Triethylenetetramine (TETA), Tetraethylenepentamine (TEPA), Hexamethylenediamine (HMDA) m-Xylenediamine, 1,3-Bis(aminomethyl)cyclohexane, Isophorondiamime (IPD), tetraethylenepentamine (TEPA), pentaethylenehexamine (PEHA), N-aminoethylpiperazine (N-AEP), isophorone diamine (IPDA), 1.3-cyclohexanebis (methylamine) (1,3-BAC); 4,4′-methylenebis(cyclohexylamine) (PACM), xylylenediamine (MXDA), n-aminoethylpiperazine, Menthanediamine alone or mixtures thereof.

12. The process as claimed in claim 4, wherein said Phenalkamine A or B of step (a) is prepared by process comprising the steps of:
a) heating the mixture of distilled cashew nut shell liquid i.e. cardanol with polyamine to afford reaction mixture;
b) adding aldehyde into the reaction mixture of step (a) when temperature of the reaction mixture reaches 70° C. to 80° C. followed by stirring at the same temperature for 3 to 4 hours; and
c) removing water of condensation by raising temperature of reaction mixture to 150° C. to afford the Phenalkamine product.

13. The process as claimed in claim 12, wherein said aldehyde of step (a) is selected from paraformaldehyde, acetaldehyde, furfuraldehyde alone or in combination thereof.

14. The process as claimed in claim 12, wherein said polyamine of step (a) is selected from aliphatic, aminoalkyl, aromatic, alicyclic polyamine alone or mixture thereof.

15. The process as claimed in claim 14, wherein said polyamine of step (a) is selected from Ethylene diamine (EDA), Diethylene triamine (DETA), Triethylenetetramine (TETA), m-Xylenediamine, 1,3-Bis(aminomethyl)cyclohexane, Isophorondiamime (IPD), n-aminoethylpiperazine, Menthanediamine alone or mixtures thereof.

16. The process as claimed in claim 12, wherein mole ratio of said polyamine to cardanol is within the range of 1:1 to 5:1.

17. The process as claimed in claim 12, wherein mole ratio of said polyamine to aldehyde compound is within the range of 1:1 to 1:4.

18. A coating composition comprising:
a) the phenalkamide curing agent of general formula (I) of claim 1;
b) an epoxy resin having on average more than one glycidyl group per molecule; and
c) pigments.

19. The composition as claimed in claim 18, wherein said epoxy resin is selected from bisphenol A diglycidyl ether, bisphenol F diglycidyl ether, resorcinol diglycidyl ether, or aliphatic poly functional epoxy resins selected from glycol diglycidyl ether, neo-pentyl diglycidyl ether, hexane diglycidyl ether, tri methyl glycidyl ether triglycidyl of trimethalol propane, alone or combination thereof.
